(19) United States
(12) Patent Application Publication
(10) Pub. No.: US 2025/0261861 A1
(43) Pub. Date: Aug. 21, 2025

(54) A Data Processing System and Computer Implemented Method for Quantifying a Stenosis in a Vessel

(71) Applicant: ARTERIAX BV, Wemmel (BE)

(72) Inventors: Jeroen Sonck, Wemmel (BE); Carlos Collet, Herne (BE)

(21) Appl. No.: 18/707,395

(22) PCT Filed: Nov. 4, 2022
(86) PCT No.: PCT/EP2022/080772

§ 371 Date: May 3, 2024

(30) Foreign Application Priority Data
Nov. 4, 2021 (EP) 21206385.3

(51) Int. Cl. A61B 5/02; A61B 5/00; A61B 5/0215; G16H 50/30

(52) U.S. Cl. CPC A61B 5/02007; A61B 5/0215; G16H 50/30; A61B 5/6852; A61B 2562/0247

(57) ABSTRACT

A data processing system and computer implemented method for quantifying a stenosis in a vessel.

There is described a data processing system (30), which receives a dataset (32) comprising a set of pressure values measured during a pullback time period (40), the pullback time period (40) corresponding to the time period during which the set of pressure values were determined from measurements of a movable pressure sensor (22) while moving along a part (12) of a vessel (10). Based on a time window (42) of which the duration corresponds to a fraction of the pullback time period, there is calculated a maximum of the moving time window (42) pressure change (26) based on said dataset (32) of said part (12) of said vessel (10).

### CROSS REFERENCE TO RELATED APPLICATIONS

This application is a national phase entry under 35 U.S.C. § 371 of International Patent application PCT/EP2022/080772, filed Nov. 4, 2023, designating the United States of America and published in English as International Patent Publication WO2023/079054 on May 11, 2023, which claims the benefit under Article 8 of the Patent Cooperation Treaty to European Patent Application Serial No. 21206385.3, filed Nov. 4, 2021, the entireties of which are hereby incorporated by reference.

### FIELD OF THE INVENTION

The invention is related to the field of data processing systems and computer implemented methods for quantifying the potential presence of a stenosis, stricture, lesion, . . . in a vessel, and more particularly in a blood vessel, such as for example a coronary artery.

### BACKGROUND

A novel functional outcome index (FOI) derived from fractional flow reserve (FFR) pullback curves, and which quantifies the pattern of coronary artery disease (CAD) in such a way that it can more easily be determined whether it is either focal or diffuse is known from WO2020/212459, which describes means and devices for assessing coronary artery disease. According to an embodiment of the FOI values close to zero represent diffuse disease and values close to 1 focal CAD. In order to determine this FOI with sufficient inter-operator and intra-operator reproducibility there was made use of precise positional information and therefor it was preferred to obtain the FFR curves from motorized pullbacks or to take great care of positional accuracy, for example by means of medical imaging, when making use of manual pullbacks. This requires additional hardware in the Cath lab, and leads to prolonged adenosine infusions during the pullback operation leading to long procedural times and patient discomfort.

There is a need for a simpler, more efficient, robust and reliable system for quantifying the potential presence of a stenosis, stricture, lesion, . . . in a vessel, which provides for a sufficient inter-operator and intra-operator reproducibility.

### SUMMARY

According to a first aspect of the invention, there is provided a data processing system comprising a processor, configured to:
receive a dataset comprising a set of pressure values measured during a pullback time period, the pullback time period corresponding to the time period during which the set of pressure values were determined from measurements of a movable pressure sensor while moving along a part of a vessel;
determine a time window of which the duration corresponds to a fraction of the pullback time period;
calculate the maximum of the moving time window pressure change based on said dataset of said part of said vessel.

Surprisingly, in this way, a robust and simple system is provided for evaluating the presence and characteristics of a stenosis in a vessel, which only requires a dataset of pressure values along the part of the vessel at interest, measured during a particular time period, without the need for further data such as the location of the measured pressure values along the vessel, or specific or complex measurement devices for generating the dataset. Any suitable system comprising a pressure sensor that can be moved along the part of the vessel at interest during the pullback time period, is able to generate the dataset for this data processing system. Such sensor systems, comprising for example a catheter with a pressure wire, are generally available, as well as data processing systems that are able to generate and/or log a data set with pressure values generated during a pullback of such a pressure wire, during which pressure values are recorded across the part of the vessel of interest. The system is able to provide for a surprisingly robust and reproducible parameter, even when for example the data set generated by means of a manual pullback of such a pressure wire. Surprisingly, the data processing system provides for a robust processing of the dataset, which provides for a high intra-operator and inter-operator reproducibility, even in the face of intra-operator and inter-operator variations with respect to the length of the pullback time period and the boundaries of the predetermined part of the vessel. Further this data processing system, and its requirements for only simple dataset, allows for a simple and efficient measurement operation for generating the data, thereby reducing the time needed for generating the data for the data processing apparatus, which reduces the time period and the delay involved in obtaining such measurements. This is particularly useful when for example the pressure measurements are obtained during hyperaemia, or in other words under hyperaemic conditions, as a reduced time period for obtaining the pressure measurements, provides for a shorter time period and reduced impact of the effect of a hyperaemic agent, as the time period during which, a stable and continuous hyperaemia must be achieved is reduced. Although alternative embodiments are possible, such measurements under hyperaemic conditions offer the advantage of a more reliable and reproducible pressure measurements during a pullback operation generating a pullback curve, when compared to alternative methods, such as for example making use of pressure measurements under resting conditions or other non-hyperaemic conditions. According to a preferred embodiment, the pressure measurements are performed under steady state maximal hyperaemia.

According to an embodiment, there is provided a data processing system, wherein the set of pressure values of the dataset, comprises:
pressure values determined from measurements of said movable pressure sensor while moving along said part of said vessel during said pullback time period;
optionally, pressure values determined from measurements of a stationary pressure sensor which remains stationary in said vessel during said pullback time period;
optionally, pressure values determined from a ratio of said pressure values of said movable pressure sensor and said stationary pressure sensor during said pullback time period; and
optionally, a time-reference comprising:
   an indication of the time during the pullback time period of the pressure measurements related to the set of pressure values;
   an indication of a measurement frequency and/or a measurement interval of the pressure measurements related to the set of pressure values.

The pressure values of the dataset could correspond to the pressure measurements of the movable sensor, or to a suitable value calculated from these pressure measurements during the pullback time period, such as for example a suitable moving mean, average, etc. value, which filters out rhythmic pressure variations, such as for example caused by the heartbeat cycle, etc, which are not caused by a stenosis, stricture, lesion, etc. of the vessel. According to preferred embodiments there is also made use of measurements of a stationary pressure sensor to allow for a reference measurement, which for example compensates for low frequency pressure variations in the vessel during the pullback time period, which are not related a stenosis, stricture, lesion, etc. of the vessel, but may influence the pressure measurement of the movable pressure sensor. In this way a more precise determination of the maximum pressure change associated with the moving time window is possible. According to a preferred embodiment, this can be implemented in a simple and efficient way, by making use of ratio of the pressure values of said movable sensor and said stationary sensor.

According to an embodiment, there is provided a data processing system, further configured to:
determine the duration of the time window in the range of 5% up to and including 50% of the pullback time period, preferably in the range of 10% up to and including 40%, for example in the range of 15% up to and including 30%, for example 20%.

According to a preferred embodiment the duration of the pullback time period is in the range of:
at least 10 s, preferably at least 15 s, more preferably at least 20 s; and at most 500 s, preferably at most 400 s, more preferably at most 300 s; and/or
at least 10 heartbeat cycles, preferably at least 15 heartbeat cycles, more preferably at least 20 heartbeat cycles; and at most 500 heartbeat cycles, preferably at most 400 heartbeat cycles, more preferably at most 300 heartbeat cycles.

According to a preferred embodiment, there is provided a data processing system, further configured to:
determine the duration of the time window in the range of:
   at least 1 s, preferably at least 3 s, more preferably at least 5 s; and at most 100 s, preferably at most 30 s, more preferably at most 15 s; and/or
   at least 1 heartbeat cycle, preferably at least 3 heartbeat cycles, more preferably at least 5 heartbeat cycles; and at most 100 heartbeat cycles, preferably at most 30 heartbeat cycles, more preferably at most 15 heartbeat cycles.

Such a relative duration of the time window with respect to the overall duration of the pullback time period surprisingly has provided for a simple, efficient parameter to quantify the presence of a stenosis, stricture or lesion in a vessel, which has reduced resistance to inter-operator and intra-operator variation when performing a pullback operation, and which has less strict requirements with respect to the precision of the position of the start and end position of the pullback operation.

According to an embodiment the data processing system is further configured to:
calculate from said time-referenced dataset the pressure change associated with the pullback time period; and
calculating a ratio of:
   said maximum pressure change associated with said time window; and
   said pressure change associated with the pullback time period,
      thereby determining the contribution of the pressure change during the time window with respect to the pressure change during the pullback time period.

In this way the impact of the section of the vessel with the worst impact on the pressure values by a suspected stenosis, stricture, lesion, . . . with respect to the overall impact of all potential manifestations of a stenosis, stricture, lesion, etc. in the vessel during the pullback operation is quantified in an efficient, robust, simple and reliable manner.

According to an embodiment the data processing system is further configured to:
determine a threshold for a rate of change of the pressure values of said dataset in function of time;
determine, based on said dataset a portion of the pullback time period associated with said dataset in which the rate of change is equal to or larger than said threshold.

In this simple and robust way it can be determined what portion of the part of the vessel investigated during the pullback maneuver suffers from lesions, strictures, stenosis, . . . without the need for any precise positioning and with a good inter-operator and intra-operator reproducibility.

Preferably the pressure values, are relative pressure values as determined from a ratio of said pressure values of said movable pressure sensor and said stationary pressure sensor during said pullback time period.

According to an embodiment the data processing system is further configured to:
determine the threshold as a relative pressure rate of change in the range of at least 0.0005 per one second or per 1/100th of the pullback time period, preferably in the range of at least 0.0010, for example equal to 0.0015.

According to an embodiment the data processing system is further configured to:
determine the threshold as a rate of change of the pressure values in the range of at least 0.05% of a maximum pressure value per subsection of the pullback time period, preferably in the range of at least 0.10%, for example equal to 0.15%, wherein the subsection is for example:
one second; or
1/100th of the pullback time period.

According to an embodiment the maximum pressure value could for example be determined as the maximum of the pressure values determined during the pullback time period, or a predetermined maximum pressure value.

According to an embodiment the data processing system is further configured to:
calculate a functional outcome index (FOI) based on the combination of:
   said contribution of said maximum pressure change associated with said time window, with respect to said pressure change associated with the pullback time period; and
   said portion of the pullback time period associated with said dataset in which the rate of change is equal to or larger than said threshold.

Such a functional outcome index can be calculated based on pressure, without any need for any positional references, thereby enabling this index to be calculated in circumstances where only limited resources are available and/or when it is desired or required to determine the index in a fast and efficient way, for example, when it is important, for example during an urgent intervention or an urgent or risky phase of an intervention, to reduce the time for obtaining the pressure data and/or where any additional time needed to ascertain precise positional references would lead to undesired risks. Such a functional outcome index enables an efficient quantification of both the severity of the impact of the stenosis, stricture, lesion, . . . as well as how extensive the presence of the stenosis is spread along the part of the vessel along which the pressure sensor moves during the pullback operation.

According to an embodiment the data processing system is further configured to:
calculate said functional outcome index (FOI) based on the formula:

$$FOI = \frac{\begin{matrix}\frac{maximum pressure change ( time window )}{pressure change ( pullback time period )} + \\ ( 1 - threshold exceeding portion )\end{matrix}}{2}$$

wherein:
maximum pressure change (time window) is defined said the maximum pressure change associated with said time window;
pressure change (pullback time period) is defined as said pressure change associated with the pullback time period; and
threshold exceeding portion is defined as said portion of the pullback time period associated with said dataset in which said rate of change is equal to or larger than said threshold.

This allows for an index in which both the impact and the extent of stenosis, stricture or lesion in the vessel are provided with similar weight.

According to an embodiment there is provided a data processing system, wherein
said set of relative pressure values comprises a fractional flow reserve (FFR) pullback curve comprising FFR values determined from measurements of the movable pressure sensor with respect to a stationary pressure sensor during the pullback time period, and wherein the data processing system is further configured to:
calculate said functional outcome index (FOI) based on the formula:

$$FOI = \frac{\begin{matrix}\frac{maximum Δ FFR ( time window )}{Δ FFR ( pullback time period )} + \\ ( 1 - ( threshold exceeding portion ) )\end{matrix}}{2}$$

wherein:
maximum Δ FFR (time window) is defined as the maximum difference between FFR values associated with the start and the end of said time window;
Δ FFR (pullback time period) is defined as the difference between FFR values associated with the start and the end of said pullback time period.
threshold exceeding portion is defined as said portion of the pullback time period associated with said dataset in which said rate of change in FFR is equal to or larger than said threshold.

Such an embodiment is especially useful for quantifying the patterns of coronary artery functional disease in a coronary vessel from a patient under hyperaemic conditions. Under such conditions there can be generated pressure values that represent an FFR pullback curve during a pullback operation. Determining such an FFR pullback curve is done by determining FFR values from measurements of the movable pressure sensor, also referred to as distal pressure or Pd, with respect to a stationary pressure sensor, also referred to as proximal or aortic pressure Pa, during the pullback time period. It is clear that for example the stationary pressure sensor is positioned at the ostium of the vessel and that the movable pressure sensor, during the pullback time period is moved between a more distal part of the vessel, for example the most distal part of the vessel, or a part of the vessel distal of a suspected stenosis, stricture or lesion, and the ostium of the vessel. When such measurements are for example performed under hyperaemic conditions in a coronary artery, then these values that are determined based on these measurements during the pullback time period, during which the movable sensor is moved along the vessel, are referred to as FFR values of an FFR pullback curve, and are typically determined as the ratio of Pd/Pa, wherein Pd and Pa could for example be determined from the measured pressure values after any suitable form of pre-processing such as for example by means of a moving mean or average function which is configured to filter out the rhythmic and/or periodical component of the heartbeat cycle. According to particular embodiments, the pre-processing of these FFR values, or any similar measured pressure values as described herein, could for example include a moving mean or average function with a time window in the range of one up to and including ten heartbeat cycles, for example one, two, or three heartbeat cycles. It is thus clear that according to a preferred embodiment FFR could for example be defined as the ratio of mean or average distal coronary pressure, which is the pressure measured by the movable pressure sensor, and the mean or average aortic pressure, which for example is the pressure measured by the stationary pressure sensor, measured during, preferably maximal, hyperaemia that is preferably achieved through administration of a potent vasodilator such as for example adenosine, ATP or papaverine either by IV infusion or by intracoronary (IC) bolus injection. Under such conditions of hyperemia, by rendering myocardial microvascular resistance constant and minimal, the impact of disease in the epicardial conduit artery on myocardial blood flow is more advantageously separated out.

According to an embodiment, there is provided a data processing system further configured to:
calculate said functional outcome index (FOI) such that the FOI is an expression of at least one of the following functional patterns of coronary artery disease:
   a focal coronary artery disease;
   a diffuse coronary artery disease.

According to an embodiment, there is provided a data processing system, wherein, the data processing system is configured to output the value of the FOI such these values quantify at least one of the following functional patterns of coronary artery disease:
the functional pattern of a focal coronary artery disease when the value is higher than 0.7;
the functional pattern of a diffuse coronary artery disease when the value is lower than 0.4; and/or
functional pattern of a mixed coronary artery disease when the value is between 0.4 and 0.7.

In this way an efficient, robust and simple index is provided, which can be used by a physician to make a suitable diagnose or treatment plan in an efficient, robust and reliable way based on the quantification of the functional pattern of the coronary artery disease.

According to a second aspect, there is provided a system comprising the data processing system according to the first aspect, wherein the system further comprises a data acquisition system coupled to and/or comprised in said data processing system, the data acquisition system configured to:
receive as input said pressure measurements from the movable pressure sensor when moved along said part of said vessel during the pullback time period wherein the movable pressure sensor is moved by means of at least one of:
   a manual pullback of a pressure wire;
   a motorized pullback of a pressure wire; and
generate and/or provide to the data processing system said set of pressure values of said dataset based on said pressure measurements during the pullback time period; and optionally, a time-reference comprising:
   an indication of the time during the pullback time period of the pressure measurements related to the set of pressure values;
   an indication of a measurement frequency and/or a measurement interval of the pressure measurements related to the set of pressure values.

According to an embodiment there is provided a system, wherein the data acquisition system further comprises at least one of the following comprising at least the movable pressure sensor and optionally a stationary pressure sensor:
A catheter comprising a pressure wire;
A catheter comprising a pressure wire configured for manual pullback along a predetermined part of a vessel during the pullback time period;
A catheter comprising a pressure wire configured for manual pullback along a predetermined part of a coronary vessel during the pullback time period; and/or
A catheter comprising a pressure wire coupled to a motorized device with a fixed pullback speed during the pullback time period.

In this way, especially, when use is made of a manual pullback operation, surprisingly, there can be determined a reliable and robust functional outcome index in a fast, efficient, simple and reliable manner, which is sufficiently robust against inter-operator and intra-operator variation of the pullback operation. Further, as there is no need for precise positional indicators to determine the functional outcome index the pullback operation can be performed with minimal delays and in a shorter time period, whether it is performed manually or with a motorized device.

According to a third aspect, there is provided a computer-implemented method for operating the data processing system according to the first aspect, wherein the method comprises the following steps performed by the data processing system:
receiving a dataset comprising a set of pressure values measured during a pullback time period, the pullback time period corresponding to the time period during which the set of pressure values were determined from measurements of a movable pressure sensor while moving along a part of a vessel;
determining a time window of which the duration corresponds to a fraction of the pullback time period;
calculating the maximum of the moving time window pressure change based on said dataset of said part of said vessel, and
   wherein optionally:
the set of pressure values of the dataset relate to pressure measurements for a coronary vessel from a patient under hyperaemic conditions, and wherein optionally the method comprises the further step of:
quantifying the patterns of a coronary artery functional disease in a coronary vessel from a patient under hyperaemic conditions.

It is clear that further alternative embodiments and/or combinations are possible, in particular further embodiments of the second and third aspect similar to the embodiments of the first aspect.

According to a fourth aspect, there is provided a computer program comprising instructions which, when the program is executed by a computer, cause the computer to carry out the method of the third aspect.

According to a fifth aspect, there is provided a computer-readable storage medium comprising instructions which, when executed by a computer, cause the computer to carry out the method of the third aspect.

### BRIEF DESCRIPTION OF THE DRAWINGS

Exemplary embodiments will now be described with respect to the drawings, in which:

FIGS. 1 to 3 schematically show an embodiment of a data processing system at different times during a pullback time period of a pullback operation;

FIG. 4 schematically shows an embodiment of absolute pressure values received by a data processing system similar as shown in FIGS. 1 to 3;

FIG. 5 schematically shows an embodiment of relative pressure values and changes thereto as determined by the data processing system, based on the pressure values of the embodiment of FIG. 4;

FIG. 6 schematically shows an embodiment of a computer-implemented method;

FIGS. 7 and 8 show a comparison of experimental values between a known location based functional outcome index reference formula and an embodiment of the new time based FOI using a sample of motorized pullbacks;

FIGS. 9 and 10 show a flowchart of patient inclusion in the experiment;

FIG. 11 shows the outline of the experiment in relation to the cohorts;

FIGS. 12 and 13 shows baseline clinical and procedural characteristics stratified by cohort;

FIG. 14, shows a table with a statistical analysis of experimental data similar to the table of FIG. 7.

FIG. 15 shows a comparison of experimental data similar as shown in FIG. 8 for the experiment of FIG. 14.

FIG. 16 shows a case example of the analysis in FIGS. 14 and 15; which shows a similar representation as the embodiment of FIGS. 4 and 5; and

FIGS. 17 to 22 show further statistical analysis and case examples of experimental data;

FIG. 23 shows an embodiment of a suitable computing system for use as a data processing system.

### DESCRIPTION

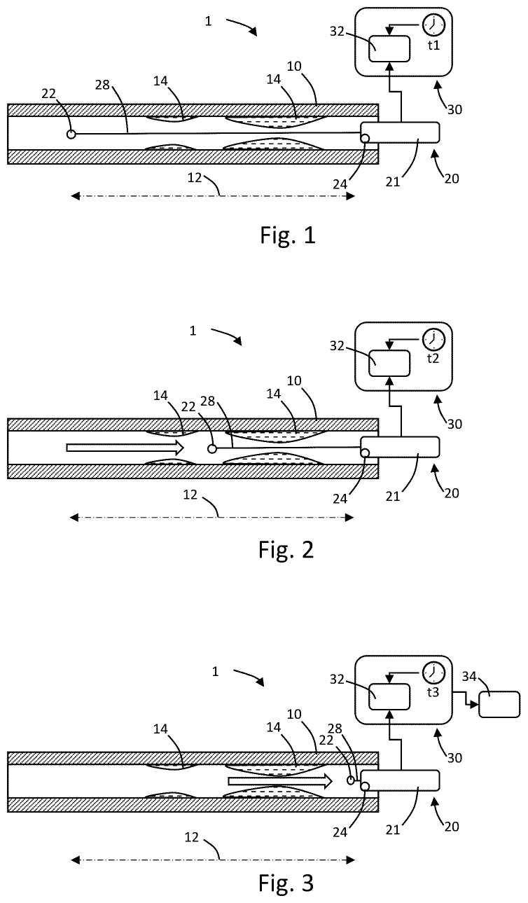

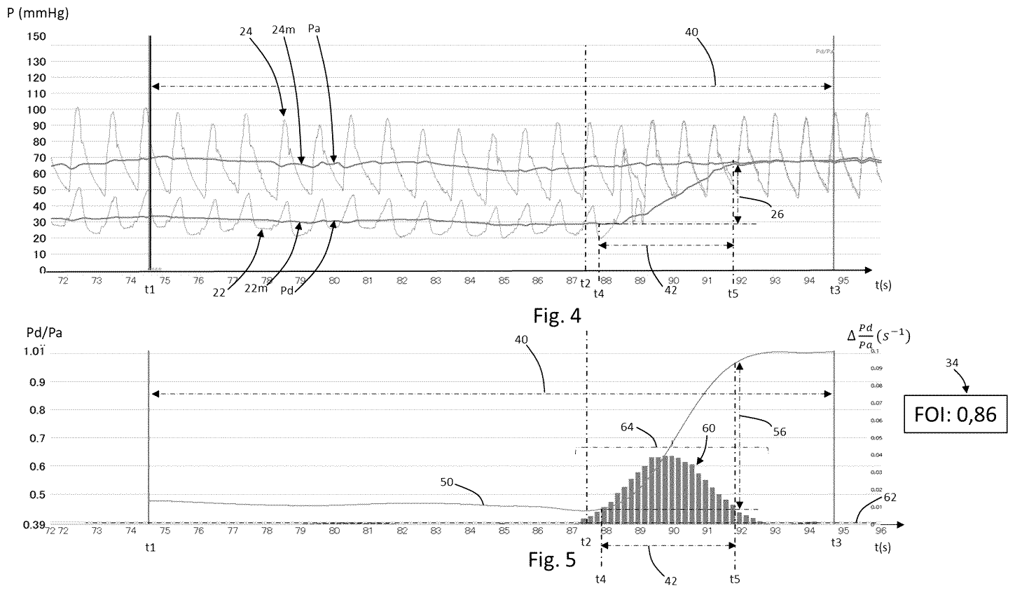

FIG. 1 shows an embodiment of a data processing system 30. As will be described in further detail below, the data processing system 30 preferably comprises a suitable processor and any other suitable data processing components, such as memory etc. which allow it to perform suitable data processing operations. As shown, the data processing system 30 is configured to receive a dataset 32 comprising pressure values measured during a pullback time period. The pullback time period 40, as shown in Further detail below with respect to FIG. 4 corresponds to the time period during which a set of pressure values is measured by means of a movement of a pressure sensor 22 moving along a vessel 10. Such a vessel 10, may for example be a coronary artery according to the embodiment shown, or any other suitable vessel, such as for example any blood vessel or other tubular organ or structure, preferably in which there is risk for the occurrence of a stenosis, stricture, lesion, etc. As shown, in FIG. 1 to FIG. 3, during the pullback time period 40, the pressure sensor 22 may move along a predetermined part 12 of the vessel 10. FIG. 1 shows the start of the pullback time period 40, schematically referenced as t1. As shown as the pullback time period 40 progresses, the pressure sensor 22 is moved along the vessel 10 in the direction indicated with the arrow in FIG. 2, which is typically in a direction against the direction of the flow of fluid in the vessel, such as for example in a direction against the direction of the blood flow in a coronary artery, however it is clear that alternative embodiments are possible in which the direction of movement has a different relationship with direction of the flow of the fluid in the vessel. FIG. 2 schematically shows the progression of the pullback time period 40, which is schematically referenced as t2, which corresponds with a time later than t1, but still within the pullback time period 40. FIG. 3 schematically represents the end of the pullback time period 40, schematically represented as t3, during which, as shown, the pressure sensor 22 has moved further along the part 12 of the vessel 10 along the same direction as schematically represented by means of the arrow. It is thus clear that, during the pullback time period, the pressure sensor 22 is moved in the vessel 10, so that it travels along at least a part of the length of the vessel, which will be referenced as a predetermined part 12 of the vessel 10 in the context of this description.

According to an embodiment the vessel 10 is for example an artery in which a catheter 20 is inserted. According to such an embodiment the catheter 20 for example comprises a sheath 21 and a guidewire 28. The guidewire 28 comprises a tip to which a pressure sensor 22 is mounted for measuring the pressure of the fluid in the vessel, in this embodiment the pressure in the blood. As shown in FIG. 1, at the start of such a pullback operation, the tip of the guidewire 28 with pressure sensor 22 is positioned at a distance from the sheath 21 of the catheter 20. Subsequently this distance is reduced during the pullback operation, such as for example shown in FIG. 2 and subsequently FIG. 3 by pulling the guidewire 28 in a direction indicated with the arrow, so that the tip of the guidewire 28, moves closer to the tip of the sheath 21 of the catheter 20. Such a movement of the guidewire 28 and the pressure sensor 22 mounted at its tip is caused by an operation that can be referred to as pulling the guidewire 28 through the sheath 21 of the catheter 20, as is well known to a person skilled in the art. During such a pullback operation, as shown in FIGS. 1-3, it is clear that the pressure sensor 22 is configured to move along a predetermined part 12 of the vessel 10. This part 12 of the vessel 10, which is associated with the pullback operation, is determined by the position of the pressure sensor 22 at t1, the start of the pullback operation, and by the position of the pressure sensor 22 at t3, the end of the pullback operation. It is clear that there is no need for any precise positional reference, with respect to this start and end position of the part 12 of the vessel 10 along which the pressure sensor 22 travels during the pullback operation. As will be explained in more detail below, all that is required for the system of the invention is an indication of the start time t1 and the end time t3 of the pullback operation.

As shown, according to an embodiment in which the data processing system 30 enables measurements that are associated with a potential blockage or restriction of the flow in the vessel, such as for example the blood flow in a coronary vessel, or the flow of any other fluid in any other suitable vessel, the pullback operation may start at t1 in such a way that the pressure sensor 22 is positioned, downstream or distal of a suspected stenosis 14 or stricture in the vessel 10. The pressure measured by the pressure sensor 22 is typically also referred to as the distal pressure or Pd. According to the embodiment shown, the pullback operation is started at t1, by moving the pressure sensor 22, for example by means of the guidewire 28, upstream along the vessel 10, until the suspected stenosis 14 has been transgressed and the pullback operation is ended at t3.

As shown in FIG. 4, at least during this pullback time period 40 the pressure sensor 22 measures the pressure of the fluid flow in the vessel 10. The y-axis shows the absolute pressure measured by pressure sensor 22 in mmHg and the x-axis shows a time-reference for the pressure data measured during the pullback time period in seconds. As shown in, the start time t1 is referenced as about 74.5 s and the end time t3 is referenced as 94.8 s. The duration of the pullback time period 40 according to this embodiment is about 20 s. As shown in FIGS. 1-3 the pressure sensor 22 is schematically coupled to the data processing system 30 in such a way that based on the measurements of the pressure sensor 22 during the pullback time period 40 of the pullback operation a dataset 32 is generated comprising this set of pressure values measured by pressure sensor 22 during the pullback operation. It is clear that alternative durations for the pullback time period 40 are possible, as will be explained in further detail below.

According to the embodiment shown, in addition to the measurements measured by pressure sensor 22 moving along the part 12 of the vessel 10 associated with the pullback time period 40, there is also a further pressure sensor 24 arranged at or near the tip of the sheath 26 of the catheter 20. This pressure sensor 24 remains stationary during the pullback time period 40 and is configured to measure the pressure of the fluid flow upstream of the part 12 of the vessel 10 along which the pressure sensor 24 moves during the pullback time period 40. Or in other words, this pressure sensor 24 is configured to measure the pressure in the vessel 10 upstream of or proximal to a suspected stenosis 14 or stricture in the vessel 10. The pressure measured by the pressure sensor 24 is typically also referred to as the proximal pressure or aortic pressure Pa.

As shown in the embodiment of FIG. 4, the pressure measurements of the pressure sensor 22 are processed in such a way that a mean pressure 22m is calculated from the pressure measurements of the pressure sensor 22, which reduces the variation in the pressure signal caused by the different phases of a heartbeat cycle. As shown, similarly also the pressure measurements of the pressure sensor 24 are processed such that a mean pressure 24m is calculated from the pressure measurements of the pressure sensor 24. It is clear that alternative embodiments, in which any other suitable type of pre-processing of the pressure measurements from a pressure sensor 22, 24 are performed, such as for example a moving or rolling mean, average, variation, minimum, maximum, etc. are possible. According to further alternative embodiments, no pre-processing of the pressure measurements is desired. This preprocessing of the pressure measurements, according to the embodiment shown in FIG. 4, could for example be performed by the data processing system 30. According to particular embodiments, the pre-processing of these pressure measurements, or any similar measured pressure values as described herein, could for example include a moving mean, average, . . . function with a time window in the range of one up to and including ten heartbeat cycles, for example one, two, or three heartbeat cycles.

According to the embodiment shown, the dataset 32 comprises both the measurements of pressure sensors 22, 24 and the pre-processed data of the mean pressure measurements 22m, 24m. However, it is clear that alternative embodiments are possible, in which the dataset 32 comprises only pressure measurements with or without preprocessing, as long as in general the dataset 32 comprises a set of pressure values measured during a pullback time period 40 during which pressure values were determined by means of a movement of a pressure sensor 22 along a part of a vessel 10. In other words any pressure values, directly and/or indirectly measured, derived and/or calculated from measurements of a pressure sensor 22 moving along the part 12 of a vessel 10 during a pullback operation.

The data processing system 30 will subsequently determine a time window 42 of which the duration corresponds to a fraction of the pullback time period. According to the embodiment shown, the time window 42 could for example be determined as 20% of the pullback time period 40. Or in other words for the embodiment shown in FIGS. 4 and 5, of which the pullback time period is about 20 s, the time window 42 could for example be about 4 s, or in other words a fraction of 20% of the pullback time period 40. It is clear that according to alternative embodiments, another suitable fraction of the time period 40 is possible as will be described in further detail below. It is clear that this fraction can for example be determined by an operator, for example by means of a suitable input that is stored as a suitable setting or configuration for the data processing system 30. However, alternative embodiments are possible, in which for example the fraction is determined or set in any other suitable way, manually and/or automatically.

Based on the dataset 32 associated with the part 12 of the vessel 10 along which the pressure sensor 22 was moved, the data processing system 30 will then calculate the maximum of the moving time window pressure change. In other words, according to the embodiment shown in FIGS. 1-5, based on the dataset 32 associated with pressure measurements of pressure sensor 22 moved along the part 12 of vessel 10 during the pullback time period 40 starting at t1 and ending at t3, it is determined for which time window, the pressure change is maximum. When according to such an embodiment, the time window 42 is for example 20% of the duration of the pullback time period 40, there is for example determined the duration of the pullback time period 40, by calculating the difference between t3 and t1, and therefrom there is then determined the time window 42, to be 20% of that duration. However, it is clear, that alternative embodiments are possible for determining a suitable duration of the time window 42 with respect to the pullback time period 40 as will be described in further detail below. When for example the pullback time period 40, or t3 minus t1 is about 20 s as shown in FIGS. 4 and 5, then the time window 42 is for example about 4 s. According to the embodiment shown in FIG. 4, this means that the mean pressure 22m derived from the pressure measurements 22 of the sensor 22 is processed by means of a moving time window 42, and that the difference between the mean pressure 22m at the start of the time window 42 and at the end of the time window 42 is calculated. As shown in FIG. 4, it is detected that for the time window 42 starting at t4 and ending at t5, the difference, or in other words the pressure change 26 associated with the dataset 32 based on the mean pressure 22m of the pressure sensor 22 moving along the part 12 of the vessel 10 is at its maximum. It is clear that according to such embodiment, the dataset is based on or determined from the pressure measurements of the sensor 22 moving along the vessel 10 during the pullback time period 40.

As shown, in FIG. 5, according to a preferred embodiment, it is also possible to make use of relative pressure measurements, such as for example the ratio 50 of:
the mean pressure 22m associated with pressure measurements of movable pressure sensor 22 moving along the vessel 10 during the pullback time period 40, with respect to
the mean pressure 24m associated with pressure measurements of stationary pressure sensor 24 remaining stationary in the vessel 10 during the pullback time period 40.

According to the embodiment shown in FIG. 5, at time t1, this ratio 50 is about 0.48 as calculated from a ratio of pressure 22m, also referred to as Pd as mentioned above, of 33 mm Hg with respect to a pressure 24m, also referred to as Pa as mentioned above, of 69 mm Hg. During the pullback time period 40 this ratio remains relatively stable until time t2, where the ratio starts to climb towards a value of about 1.

This ratio 50 determined from pressure values 22m relating the measurements of the movable pressure sensor 22 with respect to pressure values 24m relating to the concurrent measurements of the stationary pressure sensor 24 during the pullback time period 40, could also be referred to as the ratio of Pd/Pa. Such a set time-referenced of pressure values 50 could also be referred to as relative pressure values or measurements, as they relate to a ratio of the measurements from a movable pressure sensor 22 to that of another stationary pressure sensor 24, which provides for a reference point for the pressure measurements of the movable pressure sensor 22. Similar, as explained above with respect to FIG. 4, as shown in FIG. 4, it is detected that for the time window 42 starting at t4 and ending at t5, the difference associated with the ratio 50 of Pd 22m with respect to Pa 24m, or in other words the change 56 associated with the relative pressure change 56 associated with the dataset 32 based on the ratio 50 of the moving mean pressure 22m of the movable pressure sensor 22 and the moving mean pressure 24m of the stationary pressure sensor 24 during the pullback time period 40 is at its maximum. It is clear that, according to such embodiments, the dataset is based on or determined from the pressure measurements of both the movable pressure sensor 22 configured to move along the vessel 10 during the pullback time period 40, and additionally the stationary pressure sensor 24, configured to remain stationary in the vessel 10 during the pullback time period 40.

It is further clear that the set of pressure values of the dataset 32 are preferably time-referenced, such as for example shown in the embodiments of FIGS. 4 and 5, in which the values of the dataset 32 are shown on the y-axis. These pressure values are for example absolute pressure values 22, 22m, 24, 24m determined from measurements of the movable pressure sensor 22 or the stationary pressure sensor 24, or relative pressure values 50 determined from a ratio of pressure values determined from the pressure measurements of these pressure sensors 22, 24. These values of the dataset 32, shown on the y-axis, are referenced to an x-axis that provides a time-reference associated with the values associated with the dataset 32. As shown, according to such an embodiment, this time-reference comprises an indication of the time during the pullback time period 40 of the pressure measurements related to the set of pressure values. According to the embodiments shown, the x-axis provides an indication of the number of seconds that have elapsed since a starting point for the creation of the dataset during which a pullback operation was performed during the pullback time period 40. The pressure data of the dataset 32 is represented as data points on the y-axis along the time-reference of the x-axis. The start and end of the pullback time period 40, according to this embodiment are represented as t1 and t3 and are for example indicated by means of suitable markers, such as represented by the vertical lines, or other suitable inputs to indicate, receive and/or store the start and end time of the pullback time period 40. It is however clear that alternative embodiments are possible in which the pressure data of the dataset 32 is for example time-referenced to the starting time of the pullback time-period, or in any other suitable way. According to still further alternative embodiments, the time-reference for the time-referenced pressure data of the dataset 32 could also be determined and/or derived from an indication of a measurement frequency and/or a measurement interval of the pressure measurements related to the set of pressure values. When for example it is known that the pressure measurements from which the dataset 32 was derived from pressure measurements with a measurement frequency of 500 Hz, then, the time-reference of the pressure values in the dataset 32 which are related to a set of consecutive measurements at this measurement frequency can be derived from that measurement frequency, in other words each pressure value in the series of the dataset 32 is spaced 2 ms apart. It is however clear that still further alternative embodiments are possible.

In the above-mentioned embodiments with respect to FIGS. 1 to 5, there has been made use of a duration of the time window 42, which is defined as a fraction of the pullback time period 40 of 20%. However, it is clear that alternative embodiments for the duration of the time window 42, for determining the maximum pressure change over such a time window 42 are possible. According to such alternative embodiments the data processing system 30 could for example be configured to determine the duration of the time window 42 in the range of 5% up to and including 50% of the pullback time period 40, preferably in the range of 10% up to and including 40%, for example in the range of 15% up to and including 30%, for example 20%. The duration of the time window 42 could for example be set manually and/or automatically, for example as a suitable input for the data processing system 30 as any suitable or desired fraction of the pullback time period 40, or any other suitable value for a suitable time period, that determines a fraction of the pullback time period 40.

In certain embodiments, the pullback time period 40 is at least 10 s, preferably at least 15 s, more preferably at least 20 s, such as at least 30 s, at least 40 s, at least at least 50 s, or at least 60 s. In certain embodiments, the pullback time period 40 is at most 500 s, preferably at most 400 s, more preferably at most 300 s, such as at most 200 s. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s. In certain embodiments, the pullback time period 40 is at least 10 s and at most 500 s. In certain embodiments, the pullback time period 40 is at least 10 s and at most 400 s. In certain embodiments, the pullback time period 40 is at least 10 s and at most 300 s. In certain embodiments, the pullback time period 40 is at least 10 s and at most 200 s. In certain embodiments, the pullback time period 40 is at least 15 s and at most 500 s. In certain embodiments, the pullback time period 40 is at least 15 s and at most 400 s. In certain embodiments, the pullback time period 40 is at least 15 s and at most 300 s. In certain embodiments, the pullback time period 40 is at least 15 s and at most 200 s. In certain embodiments, the pullback time period 40 is at least 20 s and at most 500 s. In certain embodiments, the pullback time period 40 is at least 20 s and at most 400 s. In certain embodiments, the pullback time period 40 is at least 20 s and at most 300 s. In certain embodiments, the pullback time period 40 is at least 20 s and at most 200 s. In certain embodiments, the pullback time period 40 is at least 30 s and at most 500 s. In certain embodiments, the pullback time period 40 is at least 30 s and at most 400 s. In certain embodiments, the pullback time period 40 is at least 30 s and at most 300 s. In certain embodiments, the pullback time period 40 is at least 30 s and at most 200 s. In certain embodiments, the pullback time period 40 is at least 40 s and at most 500 s. In certain embodiments, the pullback time period 40 is at least 40 s and at most 400 s. In certain embodiments, the pullback time period 40 is at least 40 s and at most 300 s. In certain embodiments, the pullback time period 40 is at least 40 s and at most 200 s. In certain embodiments, the pullback time period 40 is at least 50 s and at most 500 s. In certain embodiments, the pullback time period 40 is at least 50 s and at most 400 s. In certain embodiments, the pullback time period 40 is at least 50 s and at most 300 s. In certain embodiments, the pullback time period 40 is at least 50 s and at most 200 s. In certain embodiments, the pullback time period 40 is at least 60 s and at most 500 s. In certain embodiments, the pullback time period 40 is at least 60 s and at most 400 s. In certain embodiments, the pullback time period 40 is at least 60 s and at most 300 s. In certain embodiments, the pullback time period 40 is at least 60 s and at most 200 s.

In certain embodiments, the time window 42 is at least 5% of the pullback period 40, preferably at least 10%, more preferably at least 15%, such as 20% or at least 20%. In certain embodiments, the time window 42 is at most 75% of the pullback period 40, preferably at most 60%, more preferably at most 50%. In certain embodiments, the time window 42 is at least 5% of the pullback period 40, preferably at least 10%, more preferably at least 15%, such as at least 20%, and at most 75% of the pullback period 40, preferably at most 60%, more preferably at most 50%. In certain embodiments, the time window 42 is at least 5% of the pullback period 40, and at most 75% of the pullback period 40. In certain embodiments, the time window 42 is at least 5% of the pullback period 40, and at most 605% of the pullback period 40. In certain embodiments, the time window 42 is at least 5% of the pullback period 40, and at most 50% of the pullback period 40. In certain embodiments, the time window 42 is at least 10% of the pullback period 40, and at most 75% of the pullback period 40. In certain embodiments, the time window 42 is at least 10% of the pullback period 40, and at most 60% of the pullback period 40. In certain embodiments, the time window 42 is at least 10% of the pullback period 40, and at most 50% of the pullback period 40. In certain embodiments, the time window 42 is at least 15% of the pullback period 40, and at most 75% of the pullback period 40. In certain embodiments, the time window 42 is at least 15% of the pullback period 40, and at most 60% of the pullback period 40. In certain embodiments, the time window 42 is at least 15% of the pullback period 40, and at most 50% of the pullback period 40. In certain embodiments, the time window 42 is at least 20% of the pullback period 40, and at most 75% of the pullback period 40. In certain embodiments, the time window 42 is at least 20% of the pullback period 40, and at most 60% of the pullback period 40. In certain embodiments, the time window 42 is at least 20% of the pullback period 40, and at most 50% of the pullback period 40.

In certain embodiments, the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, at least 5 s or 5 heartbeat cycles, at least 6 s or 6 heartbeat cycles, at least 7 s or 7 heartbeat cycles, at least 8 s or 8 heartbeat cycles, at least 9 s or 9 heartbeat cycles, at least 10 s or 10 heartbeat cycles, at least 11 s or 11 heartbeat cycles, at least 12 s or 12 heartbeat cycles, or at least 15 s or 15 heartbeat cycles. In certain embodiments, the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the time window 42 is at least 1 s or 1 heartbeat cycle and at most 100 s or 100 heartbeat cycles. In certain embodiments, the time window 42 is at least 1 s or 1 heartbeat cycle and at most 50 s or 50 heartbeat cycles. In certain embodiments, the time window 42 is at least 1 s or 1 heartbeat cycle and at most 30 s or 30 heartbeat cycles. In certain embodiments, the time window 42 is at least 1 s or 1 heartbeat cycle and at most 20 s or 20 heartbeat cycles. In certain embodiments, the time window 42 is at least 1 s or 1 heartbeat cycle and at most 15 s or 15 heartbeat cycles. In certain embodiments, the time window 42 is at least 1 s or 1 heartbeat cycle and at most 10 s or 10 heartbeat cycles. In certain embodiments, the time window 42 is at least 2 s or 2 heartbeat cycles and at most 100 s or 100 heartbeat cycles. In certain embodiments, the time window 42 is at least 2 s or 2 heartbeat cycles and at most 50 s or 50 heartbeat cycles. In certain embodiments, the time window 42 is at least 2 s or 2 heartbeat cycles and at most 30 s or 30 heartbeat cycles. In certain embodiments, the time window 42 is at least 2 s or 2 heartbeat cycles and at most 20 s or 20 heartbeat cycles. In certain embodiments, the time window 42 is at least 2 s or 2 heartbeat cycles and at most 15 s or 15 heartbeat cycles. In certain embodiments, the time window 42 is at least 2 s or 2 heartbeat cycles and at most 10 s or 10 heartbeat cycles. In certain embodiments, the time window 42 is at least 3 s or 3 heartbeat cycles and at most 100 s or 100 heartbeat cycles. In certain embodiments, the time window 42 is at least 3 s or 3 heartbeat cycles and at most 50 s or 50 heartbeat cycles. In certain embodiments, the time window 42 is at least 3 s or 3 heartbeat cycles and at most 30 s or 30 heartbeat cycles. In certain embodiments, the time window 42 is at least 3 s or 3 heartbeat cycles and at most 20 s or 20 heartbeat cycles. In certain embodiments, the time window 42 is at least 3 s or 3 heartbeat cycles and at most 15 s or 15 heartbeat cycles. In certain embodiments, the time window 42 is at least 3 s or 3 heartbeat cycles and at most 10 s or 10 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 5% of the pullback period 40, preferably at least 10%, more preferably at least 15%, such as 20% or at least 20%. In certain embodiments, the pullback time period 40 is at most 500 s, preferably at most 400 s, more preferably at most 300 s, such as at most 200 s and the time window 42 is at least 5% of the pullback period 40, preferably at least 10%, more preferably at least 15%, such as 20% or at least 20%. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 5% of the pullback period 40, preferably at least 10%, more preferably at least 15%, such as 20% or at least 20%.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at most 75% of the pullback period 40, preferably at most 60%, more preferably at most 50%. In certain embodiments, the pullback time period 40 is at most 500 s, preferably at most 400 s, more preferably at most 300 s, such as at most 200 s, and the time window 42 is at most 75% of the pullback period 40, preferably at most 60%, more preferably at most 50%. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at most 75% of the pullback period 40, preferably at most 60%, more preferably at most 50%.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 5% of the pullback period 40, preferably at least 10%, more preferably at least 15%, such as at least 20%, and at most 75% of the pullback period 40, preferably at most 60%, more preferably at most 50%. In certain embodiments, the pullback time period 40 is at most 500 s, preferably at most 400 s, more preferably at most 300 s, such as at most 200 s, and the time window 42 is at least 5% of the pullback period 40, preferably at least 10%, more preferably at least 15%, such as at least 20%, and at most 75% of the pullback period 40, preferably at most 60%, more preferably at most 50%. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 5% of the pullback period 40, preferably at least 10%, more preferably at least 15%, such as at least 20%, and at most 75% of the pullback period 40, preferably at most 60%, more preferably at most 50%.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles. In certain embodiments, the pullback time period 40 is at most 500 s, preferably at most 400 s, more preferably at most 300 s, such as at most 200 s, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at most 75% of the pullback period 40, preferably at most 60%, more preferably at most 50%. In certain embodiments, the pullback time period 40 is at most 500 s, preferably at most 400 s, more preferably at most 300 s, such as at most 200 s, and the time window 42 is at most 75% of the pullback period 40, preferably at most 60%, more preferably at most 50%. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at most 75% of the pullback period 40, preferably at most 60%, more preferably at most 50%.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at most 500 s, preferably at most 400 s, more preferably at most 300 s, such as at most 200 s, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles.

In certain embodiments, the time window 42 is at least 5% of the pullback period 40, preferably at least 10%, more preferably at least 15%, such as 20% or at least 20%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles. In certain embodiments, the time window 42 is at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles. In certain embodiments, the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles.

In certain embodiments, the time window 42 is at least 5% of the pullback period 40, preferably at least 10%, more preferably at least 15%, such as 20% or at least 20%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the time window 42 is at most 75% of the pullback period 40, preferably at most 60%, more preferably at most 50%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles.

In certain embodiments, the time window 42 is at least 5% of the pullback period 40, preferably at least 10%, more preferably at least 15%, such as 20% or at least 20%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the time window 42 is at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 5% of the pullback period 40, preferably at least 10%, more preferably at least 15%, such as 20% or at least 20%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles. In certain embodiments, the pullback time period 40 is at most 500 s, preferably at most 400 s, more preferably at most 300 s, such as at most 200 s, and the time window 42 is at least 5% of the pullback period 40, preferably at least 10%, more preferably at least 15%, such as 20% or at least 20%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 5% of the pullback period 40, preferably at least 10%, more preferably at least 15%, such as 20% or at least 20%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles. In certain embodiments, the pullback time period 40 is at most 500 s, preferably at most 400 s, more preferably at most 300 s, such as at most 200 s, and the time window 42 is at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles. In certain embodiments, the pullback time period 40 is at most 500 s, preferably at most 400 s, more preferably at most 300 s, such as at most 200 s, and the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 5% of the pullback period 40, preferably at least 10%, more preferably at least 15%, such as 20% or at least 20%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at most 500 s, preferably at most 400 s, more preferably at most 300 s, such as at most 200 s and the time window 42 is at least 5% of the pullback period 40, preferably at least 10%, more preferably at least 15%, such as 20% or at least 20%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 5% of the pullback period 40, preferably at least 10%, more preferably at least 15%, such as 20% or at least 20%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at most 75% of the pullback period 40, preferably at most 60%, more preferably at most 50%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at most 500 s, preferably at most 400 s, more preferably at most 300 s, such as at most 200 s, and the time window 42 is at most 75% of the pullback period 40, preferably at most 60%, more preferably at most 50%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at most 75% of the pullback period 40, preferably at most 60%, more preferably at most 50%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at most 500 s, preferably at most 400 s, more preferably at most 300 s, such as at most 200 s, and the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 5% of the pullback period 40, preferably at least 10%, more preferably at least 15%, such as 20% or at least 20%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at most 500 s, preferably at most 400 s, more preferably at most 300 s, such as at most 200 s and the time window 42 is at least 5% of the pullback period 40, preferably at least 10%, more preferably at least 15%, such as 20% or at least 20%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 5% of the pullback period 40, preferably at least 10%, more preferably at least 15%, such as 20% or at least 20%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at most 500 s, preferably at most 400 s, more preferably at most 300 s, such as at most 200 s, and the time window 42 is at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles.

In certain embodiments, the pullback time period 40 is at least 10 s, preferably at least 15 s, more preferably at least 20 s, such as at least 30 s, at least 40 s, at least at least 50 s, or at least 60 s, and the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles, or 15 s or 15 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at most 500 s, preferably at most 400 s, more preferably at most 300 s, such as at most 200 s, and the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, or 10 s or 10 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles. In certain embodiments, the pullback time period 40 is at least 10 s, preferably at least 15 s, more preferably at least 20 s, such as at least 30 s, at least 40 s, at least 50 s, or at least 60 s, and at most 500 s, preferably at most 400 s, more preferably at most 300 s, such as at most 200 s, and the time window 42 is at least 5% of the pullback period 40, preferably at least 10%, more preferably at least 15%, such as at least 20%, and at most 75% of the pullback period 40, preferably at most 60%, more preferably at most 50%, and the time window 42 is at least 1 s or 1 heartbeat cycle, preferably at least 2 s or 2 heartbeat cycles, more preferably at least 3 s or 3 heartbeat cycles, such as at least 4 s or 4 heartbeat cycles, 5 s or 5 heartbeat cycles, 6 s or 6 heartbeat cycles, 7 s or 7 heartbeat cycles, 8 s or 8 heartbeat cycles, 9 s or 9 heartbeat cycles, 10 s or 10 heartbeat cycles or 15 s or 15 heartbeat cycles, and at most 100 s or 100 heartbeat cycles, preferably at most 50 s or 50 heartbeat cycles, more preferably at most 30 s or 30 heartbeat cycles, such as at most 20 s or 20 heartbeat cycles, at most 15 s or 15 heartbeat cycles, or at most 10 s or 10 heartbeat cycles.

In addition to the above-mentioned parameter of calculating the maximum pressure change 26, 56 associated with the moving time window 42, according to a preferred embodiment, it is also possible to calculate by means of the data processing system 30 from said time-referenced dataset 32 the pressure change associated with the pullback time period 40. It is clear that this for example means, according to the respective embodiments shown in FIG. 4 and FIG. 5, the difference of the absolute pressure change of the values 22m of the moving pressure sensor 22, or the relative pressure change of values the moving pressure sensor 22 as expressed by the ratio 50, between the start time t1 of the pullback operation and the end time t3 of the pullback operation, or in other words between the start and the end of the pullback time period 40. According to the embodiment shown in FIG. 4, Pd is about 33 mmHg at t1 and Pd is about 68 mmHg at t3, and therefor the difference associated with the pullback time period 40 is about 68 mmHg-33 mmHg=35 mmHg. However according to a preferred embodiment, there is made use of the relative pressure values of the ratio 50 shown in FIG. 5, where at t1, the ratio 50 of Pd/Pa=33/69=0.48 and where at t3, the ratio 50 of Pd/Pa=68/67=1.01, and therefor the difference of the relative pressure associated with the pullback time period is about 1.01-0.48=0.53 for the embodiment shown in FIG. 5.

According to a preferred embodiment, when both said maximum pressure change associated with said time window 42 and said pressure change associated with the pullback time period 40 are calculated by the data processing system 32, such as for example described above, there can be determined a ratio of said maximum pressure change 26, 56 associated with said time window 42 and said pressure change associated with the pullback time period 40. It is clear that from this ratio of both pressure changes there can be determined the contribution of the pressure change during the time window 42 with respect to the pressure change during the pullback time period 40.

As further shown in the embodiment of FIG. 5 the data processing system 30 is further configured to determine a threshold 62 for a rate of change 60 of the relative pressure values 50 of said dataset 60 in function of time. The exemplary scale of the relative pressure values 50 is shown on the left y-axis, while the scale for the rate of change 60 of these relative pressure values 50 in function of time is schematically shown by means of the bar graphs 60 in FIG. 5. The exemplary scale for the rate of change 60 is shown on the right y-axis. According to this embodiment, as shown the data processing system 30 determines a suitable threshold at a rate of change of 0.0015 per second. This threshold 62 could be provided as a suitable manual or automatic input for the data processing system 30. Subsequently the data processing system determines a portion 64 of the pullback time period 40 in which the rate of change 60 is equal to or larger than said threshold 62 as shown in FIG. 5. Although in the embodiment of FIG. 5, this portion 64 of the pullback time period 40 is a single contingent portion, it is clear that according to alternative embodiments, this portion 64 could be formed by the total of multiple dispersed sub-portions along the pullback time period in which the rate of change 60 exceeds the threshold 62 during the pullback time period 40. According to the embodiment shown in FIG. 5, preferably the pressure values, for which the rate of change 60 is determined, are relative pressure values 60 as determined from a ratio 50 of said pressure values 22m or Pd of said movable pressure sensor 22 and said pressure values 24m or Pa said stationary pressure sensor 24 during said pullback time period 40 as described above. In other words, the rate of change 60 relates to the rate of change of the ratio Pd/Pa and can be symbolized as ΔPd/Pa. The portion 64, could be referred to as a threshold exceeding portion 64 of the pullback time period, and could for example be expressed as a percentage or any other suitable ratio relative to the pullback time period 40. In other words, according to a preferred embodiment, the portion 64 would be expressed as the ratio of:
the aggregate of the time periods during which the rate of change 60 of the pressure values 50 exceeds the threshold 62; and
the pullback time period 40.

It is clear that alternative embodiments are possible in which the threshold is set to another suitable value, such as for example a relative pressure rate of change in the range of at least 0.0005 per second or per 1/100th of the pullback time period, preferably in the range of at least 0.0010, or any other suitable threshold that is indicative of a relevant rate of change in the relative pressure values that is representative of stenosis, stricture, lesion, . . . in the vessel.

It is further clear that according to still further alternative embodiments, instead of relative pressure values, such as illustrated by means of FIG. 5 the rate of change could also be determined by means of any other suitable pressure values, such as for example absolute pressure values, etc. as described above, which are determined from the measurements of the moving pressure sensor 22 during the pullback operation. According to such embodiments, the threshold 62 can for example be determined as a rate of change of the pressure values in the range of at least 0.05% of a maximum pressure value per subsection of the pullback time period 40, preferably in the range of at least 0.10%, for example equal to 0.15%. According to such embodiments, the subsection of the pullback time period could for example be determined as a suitable absolute subsection, such as for example one second or a relative subsection, such as for example 1/100th of the pullback time period 40. It is clear that alternative embodiments are possible in which any other suitable absolute or relative subsection is chosen, such as for example 1/50th of the pullback time period or lower, 2 seconds or lower.

According to a further embodiment, the data processing system calculates a functional outcome index or FOI based on the combination of:
the contribution of the maximum pressure change 56 associated with the time window 42 with respect to the pressure change associated with the pullback time period 40; and
said portion 64 of the pullback time period 40 associated with said dataset 32 in which the rate of change 60 is equal to or larger than the threshold 62.

According to a preferred embodiment, this contribution can for example be calculated as the following ratio:

$$\frac{maximum pressure change ( time window )}{pressure change ( pullback time period )} .$$

The “maximum pressure change (time window)” is the maximum pressure change 56 associated with said time window 42, such as for example illustrated above with reference to FIG. 5, the maximum relative pressure change 56, which is in other words the difference between the relative pressure 50 at the start and end of this time window 42, for the time window 42 where this difference is at maximum. The “pressure change (pullback time period)” is the pressure change associated with the pullback time period 40, such as for example illustrated above with reference to FIG. 5, the relative pressure change between t1 and t3, the respective start and end time of the pullback time period 40, or in other words, the difference between Pd/Pa at t1 and t3.

According to a preferred embodiment the functional outcome index (FOI) can then be based on the following formula:

$$FOI = \frac{\begin{matrix}\frac{maximum pressure change ( time window )}{pressure change ( pullback time period )} + \\ ( 1 - threshold exceeding portion )\end{matrix}}{2}$$

Such a functional outcome index is especially advantageous when the vessel is a blood vessel, and more particularly when the vessel is an artery, for example a coronary artery. According to such embodiments the functional outcome index provides a means for quantifying the impact and extent of a stenosis, stricture, lesion, and/or any other functional disease that impacts the pressure of the flow in the vessel negatively. According to such embodiments, the pressure values of FIG. 4, represent for example the following:
Pa pressure values 24m determined from measurements of a stationary pressure sensor 24 positioned proximal to a suspected stenosis, stricture, lesion, . . . in a coronary artery. Alternatively, Pa could for example be determined from measurement of the aortic pressure.
Pd pressure values 22m determined from measurements of a movable pressure sensor 22 moved along the vessel, during the pullback time period, between a more distal and proximal position with respect to a suspected lesion, for example between a most distal part and the ostium of a coronary artery.

Preferably these pressure measurements are performed under hyperaemic conditions. According to such an embodiment the relative pressure values corresponding to the ratio 50 of Pd/Pa as for example shown in FIG. 5, based on these pressure measurements 22m, 24m, can be referred to as FFR values of an FFR pullback curve, determined during a pullback time period 40 of a pullback operation during which the movable sensor 22 was moved along that part of the coronary artery. According to such an embodiment the relative pressure values Pd/Pa could be referred to as FFR values, and ΔPd/Pa could be referred to as ΔFFR. This then allows a calculation of an embodiment of the functional outcome index based on the following formula:

$$FOI = \frac{\begin{matrix}\frac{maximum Δ FFR ( time window )}{Δ FFR ( pullback time period )} + \\ ( 1 - ( threshold exceeding portion ) )\end{matrix}}{2}$$

wherein:
maximum Δ FFR (time window) is defined as the maximum difference between FFR values 50 associated with the start and the end of said time window 42;
Δ FFR (pullback time period) is defined as the difference between FFR values 50 associated with the start and the end of said pullback time period 40; and
threshold exceeding portion is defined as said portion 64 of the pullback time period 40 associated with said dataset in which said rate of change 60 in FFR 50 is equal to or larger than said threshold 62.

As for example shown in the embodiments of FIG. 3, and FIG. 5, after the pullback operation, the data processing system 30, based on the above mentioned calculations, can for example output the value 34 of the FOI such these values quantify at least one of the following functional patterns of coronary artery disease:
the functional pattern of a focal coronary artery disease when the value is higher than 0.7;
the functional pattern of a diffuse coronary artery disease when the value is lower than 0.4; and/or functional pattern of a mixed coronary artery disease when the value is between 0.4 and 0.7.

According to the embodiment shown in FIGS. 1 to 5, the FOI is for example calculated to be 0.86 based on the formula above and the pressure values shown in FIGS. 4 and 5, based on pressure measurements during a pullback time period of a pullback operation in a coronary artery vessel.

It is clear that, although for the preferred embodiment described above the pressure values of the dataset 32 are determined from pressure measurements along a part of a coronary vessel 10 from a patient under hyperaemic conditions, alternative embodiments are possible in which the pressure measurements are not determined under hyperaemic conditions.

According to the embodiment shown in FIGS. 1 to 3, it is clear that the data processing system 30 comprises a data acquisition system to receive the pressure measurements of the movable pressure sensor 22 and the stationary sensor 24. It is clear that alternative embodiments are possible in which there is provided a system comprising the data processing system 30 and a separate data acquisition system coupled thereto. It is clear that according to such embodiments the data acquisition system of system 1 receives as input the pressure measurements from the movable pressure sensor 22 when moved along said part 12 of said vessel 10 during the pullback time period 40. Preferably the movable pressure sensor 22 is moved by means a manual pullback of a pressure wire 28. However according to alternative embodiments, it is also possible to for example make use of a motorized pullback of a pressure wire 28. The data acquisition system of the system 1 then generates the set of pressure values of the dataset 32 based on the pressure measurements of the pressure sensors 22, 24 during the pullback time period 40. As already mentioned above, preferably, the data acquisition system also generates a time-reference for the pressure values of the dataset 32. It is clear that according to the embodiments shown in FIGS. 1-3, that such a data acquisition system preferably comprises a catheter 20, for example a sheath 21 of a catheter 20 or any other suitable location, on which the stationary pressure sensor 24 is arranged and a pressure wire 28 on which the movable pressure sensor 22 is arranged. It is clear that such a pressure wire 28 is configured for manual pullback along the predetermined part 12 of the vessel 10 during the pullback time period 40 of the pullback operation. Such a pullback operation preferably is a manual pullback. However, according to alternative embodiments the pullback operation could also be performed by means of a motorized pullback, such as for example by a catheter and a pressure wire coupled to a motorized device with a fixed pullback speed during the pullback time period 40.

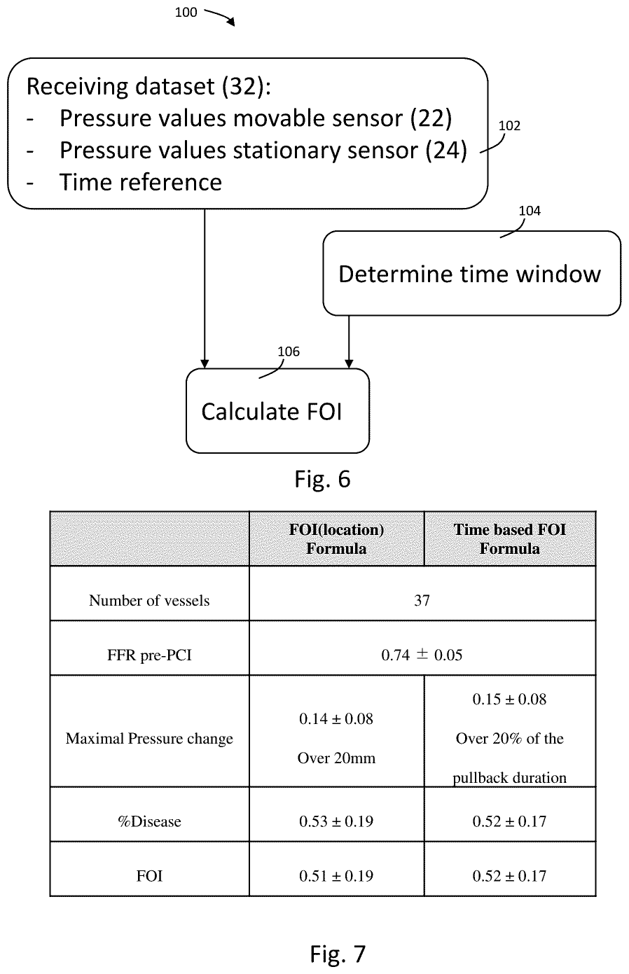

As shown in FIG. 6, there is provided an embodiment of a computer-implemented method 100 for operating the data processing system 30 as described above. The embodiment of the method 100 shown, comprises the step 102 of receiving a dataset 32 comprising a set of pressure values 22, 24 measured during a pullback time period 40 and preferably a time reference from measurements of a movable pressure sensor 22 while moving along a part 12 of a vessel 10 and a stationary pressure sensor 24. In step 104 there is then determined a time window of which the duration corresponds to a fraction of the pullback time period, for example a fraction of 20%. Then in step 106, according to this embodiment there can for example be calculated a functional outcome index, for example by means of the embodiments of the formula mentioned above. It is however clear that alternative embodiments of the computer-implemented method are possible in which a dataset 32 is determined based on measurements of a movable pressure sensor 22 during a pullback time period 40, a time window 42 is determined, and the maximum of the moving time window 42 pressure change 26 based on the dataset 32 is calculated.

### Experimental Results

There has been described an easier and more efficient approach to derive a FOI from manual pullbacks. However, up till now, the skilled person was convinced that a manual withdraw of the pressure wire is operator dependent and that reproducibility may be hampered concerning the assessment of a set of pressure values and pressure changes, also referred to as pressure gradients, determined therefrom. It is important for such a FOI that it provides for test and re-test reliability. This is of particular importance in the field of coronary physiology where cut-offs are usually applied to guide clinical decision making. While the usefulness of intracoronary pullback maneuver is increasingly recognized, up till now there was insufficient data supporting the test and re-test the reliability of such a FOI determined from manual FFR pullbacks. The current experimental results are able to: (1) to validate the described calculation of the FOI derived from manual FFR pullbacks, and compare this with the known calculation of the FOI determined from motorized FFR pullbacks as a reference, and (2) assess the intra- and inter-operator reproducibility of the described FOI derived from manual FFR pullbacks.

There was performed, a single-center study including patients with stable CAD and clinical indication for FFR interrogation. All patients underwent FFR pullback evaluations either with a motorized device or manually depending of the study cohort. Patients with ostial lesions, severe vessel tortuosity, bifurcation lesions with planned two stent strategy, difficult vessel rewiring, patients with STEMI, or severe renal dysfunction with eGFR <30 ml/min/1.73 m2 were excluded.

### Calculation of the FOI.

For the motorized pullbacks a known location-based functional outcome index FOI(location) which will be used as a reference was determined based on the following formula, which makes use of precise positional information:

$$FOI ( location ) = \frac{\begin{matrix}\frac{Maximal FFR change over 20 mm}{Vessel FFR change} + \\ ( 1 - \frac{Length with functional disease}{Vessel length} )\end{matrix}}{2}$$

The “maximal FFR change over 20 mm” is based on the magnitude of the FFR drops over a fixed length of 20 mm; and the “length with functional disease” is based on the extent of FFR deterioration, which is defined as the length, in millimeters, with an FFR drop ≥0.0015/mm. It is clear that for the calculation of this location based FOI, there is made use of absolute distance values (i.e., millimeters)

For the manual pullbacks, there is made used of the time-based formula as described above, which no longer relies on any location-based information.

$$FOI = \frac{\begin{matrix}\frac{maximum Δ FFR ( time window )}{Δ FFR ( pullback time period )} + \\ ( 1 - ( threshold exceeding portion ) )\end{matrix}}{2}$$

wherein according to this experiment the duration of the time window 42 is 20% of the pullback time period 40.

According to this experiment this formula could also be determined as:

$$FOI = \frac{\begin{matrix}\frac{Maximal FFR change over 20 \% pullback duration}{FFR change over pullback duration} + \\ ( 1 - proportion of pullback time with FFR deterioration )\end{matrix}}{2}$$

Where the “maximal FFR change or gradient over 20% of the pullback duration” calculates the FFR drop over a fixed time window of 20% of the total pullback duration. Likewise, the “percent or proportion of pullback time with FFR deterioration” reports the extent of functional deterioration, relative to the total duration of the pullback maneuver, calculated using the same FFR drop threshold of 0.0015/mm. The “FFR change over pullback duration” could also be referred to as vessel FFR change, or vessel FFR gradient.

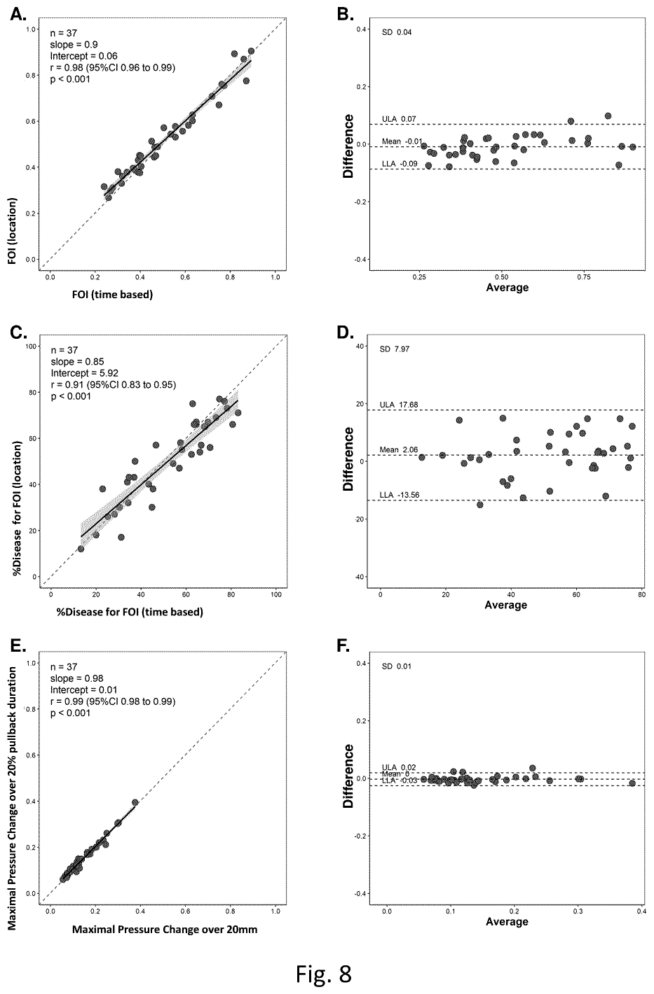

A comparison of values between the FOI(location) reference formula and the new time based FOI formula using a sample of motorized pullbacks for the known FOI(location) and manual pullbacks for the new time-based FOI is shown in FIG. 8 and the table shown in FIG. 7. The adapted formula was calculated by means of a data processing system comprising a data acquisition system in the form of a commercial console of which the software was adapted to allow for the online calculation of the FOI after a manual pullback manoeuvre. This console is manufactured by Coroventis, Coroventis Research, Uppsala, Sweden and is shown in FIG. 7. Legend of FIG. 8: FFR: Fractional Flow Reserve; PCI Percutaneous coronary intervention; FFR pre-PCI: Vessel FFR change; % Disease: for FOI(location)=Percent of vessel length with FFR deterioration; for time based FOI: Percent of pullback time with FFR deterioration; FOI: Functional Outcome Index.

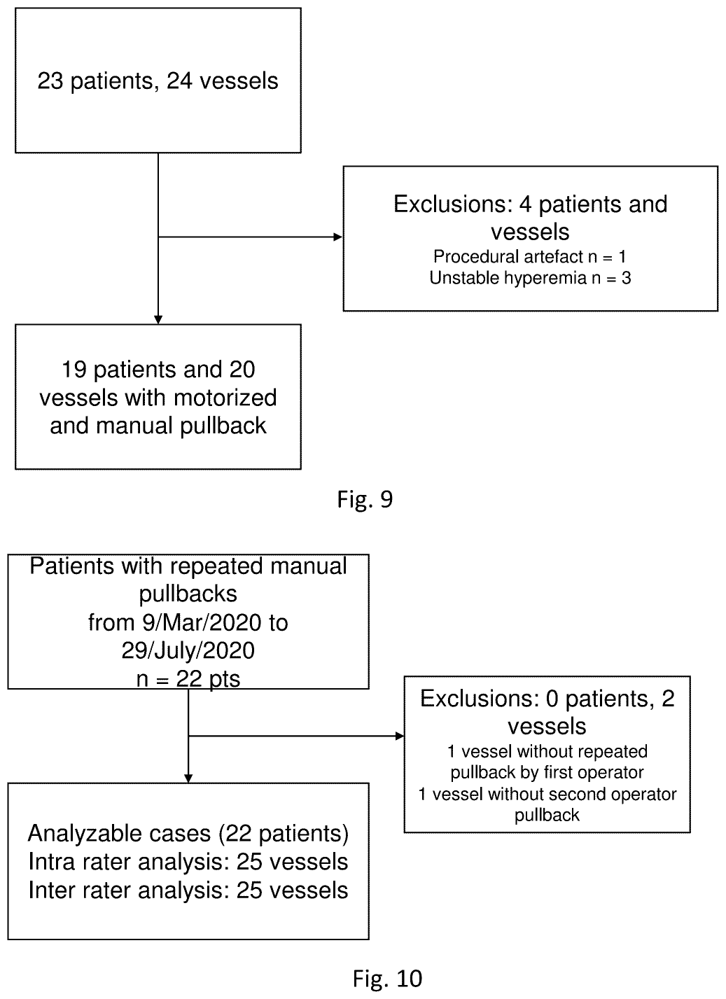

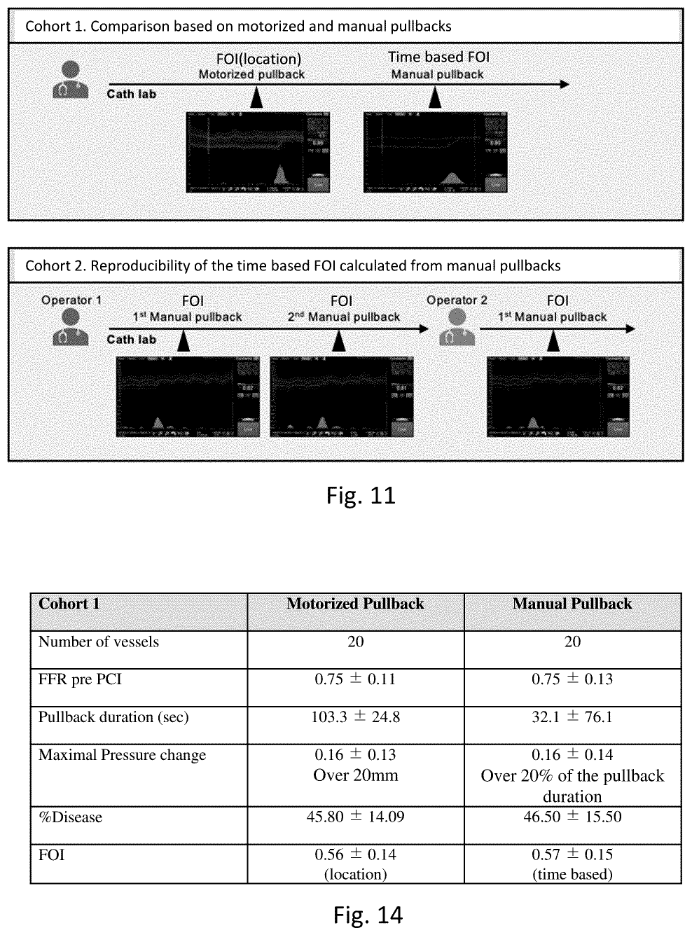

The study population of the experiment was determined by prospectively recruiting two patient cohorts. The flowchart of patient inclusion is shown in FIGS. 9 and 10. As shown in FIG. 11, the first cohort of 19 patients (20 vessels) underwent both motorized and manual FFR pullbacks to assess the agreement on values between the new time based FOI and the known FOI(location). The second cohort consisted of 22 patients (25 vessels) where repeated manual pullbacks were performed by two operators to assess the new time-based FOI inter- and intra-observer reproducibility.

### Invasive Fractional Flow Reserve Analysis

Fractional flow reserve pullback curve measurements were performed following the recommendations for the standardization of FFR measurements. The pressure wire was positioned at least 20 mm distal to the most distal coronary stenosis in vessels more than 2 mm of diameter by visual estimation. The pressure-wire sensor position was recorded using a contrast injection annotating the starting point for each pullback manoeuvre to ensure the same starting position among pullbacks. All patients received intra-coronary nitrates administration before FFR measurement. Hyperemia was induced either using adenosine at a dose of 140 μg/kg/min via a peripheral or central vein or intra-coronary papaverine at a dosage of 8 mg in the right coronary artery and a bolus of 12 milligram in the left coronary artery and flushed with saline. If FFR drift (≥0.03) was observed, the FFR measurement was repeated. FFR measurements were performed using CoroFlow version 3.5. (Coroventis Research AB, Uppsala, Sweden). All FFR tracings were then assessed by a core laboratory (CoreAalst BV, Aalst, Belgium).

The experimental results for determining the FOI(location) with motorized pullbacks were performed as follows. A pullback device (Volcano R 100, San Diego California, United States) was adapted to grip the coronary pressure wire (PressureWire X, St Jude Medical, Minneapolis, United States) and set at a speed of 1 mm/s to pullback the pressure-wire until the tip of the guiding catheter during continuous hyperemia.

The experimental results for determining the new time based FOI based on a manual pullback were performed as follows: For manual FFR pullback, the operator withdrew the pressure wire at a stable and constant, or substantially stable and constant speed for 20 to 30 seconds during stable hyperemia. When the pressure sensor 22 reached the catheter tip the recording was stopped. For determining the inter-operator agreement, the second operator remained blinded to the calculated FOI.

The following statistical analysis was performed. Continuous variables are presented as mean and standard deviation or as median and interquartile range, depending on the distribution. Categorical variables are presented as percentages and counts. Pearson correlation coefficients, intra-class correlation coefficients (ICC), Bland-Altman method, and the coefficient of variation were used to assess the FOI's reproducibility based on the experimental results. The study was directed to evaluate the reproducibility of the time based FOI derived from manual pullback operations. The sample size was calculated assuming a maximal coefficient of variation (COV) of 15% with an 11% two-sided confidence interval for the intra-observer reproducibility and a 20% maximal COV with 15% two-sided confidence interval for the inter-observer reproducibility. The sample size calculation required 20 patients with paired measurements for both the intra- and inter-operator reproducibility analyses. All analyses were performed using R (R Foundation for Statistical Computing, Vienna, Austria). COV from duplicate measurements was calculated with MedCalc Statistical Software version 19.3.1 (MedCalc Software, Ostend, Belgium).

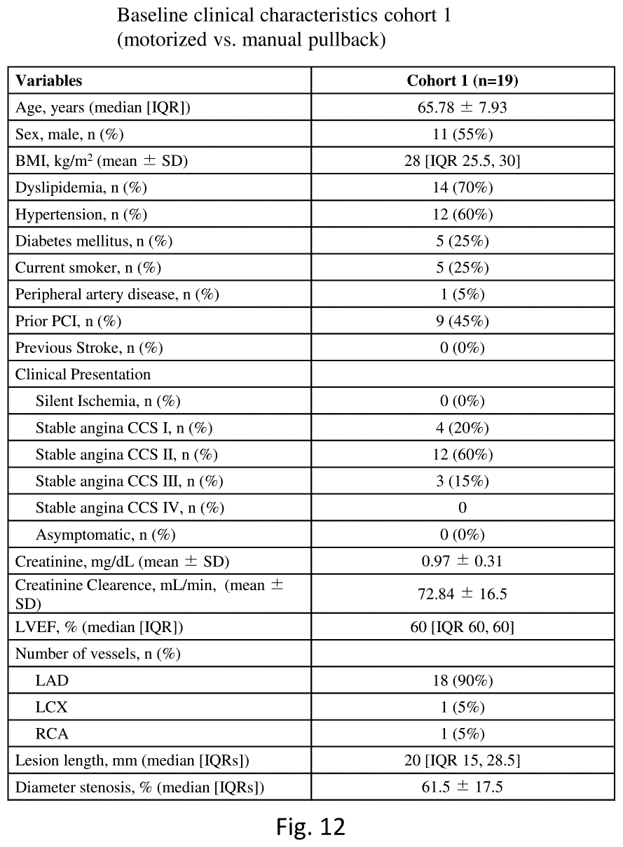

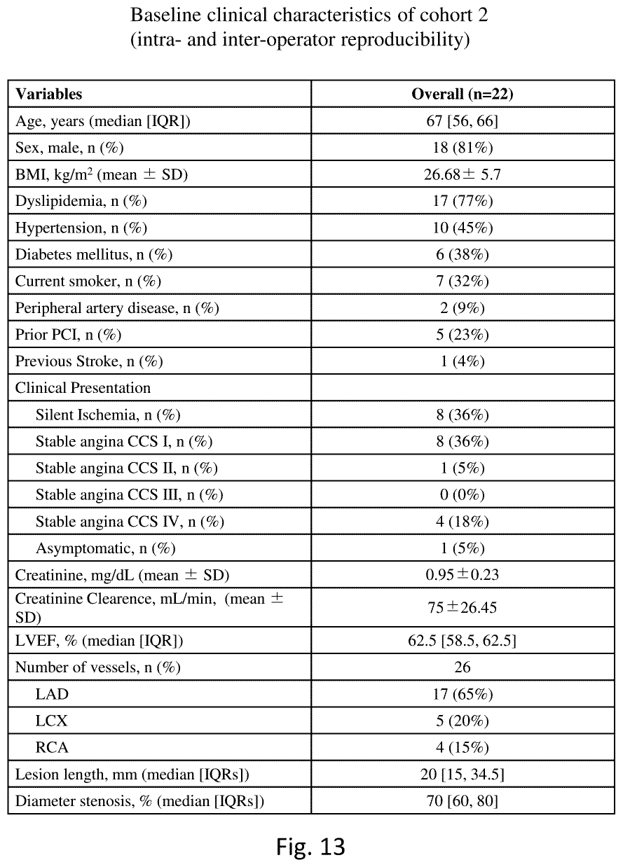

This statistical analysis led to the following results. 41 patients were included in two cohorts. The table in FIGS. 12 and 13 shows baseline clinical and procedural characteristics stratified by cohort. Legend for FIGS. 12 and 13: SD Standard deviation. BMI Body mass index. PCI Percutaneous coronary intervention. CCS Canadian Cardiovascular Society. LAD Left anterior descending. LCX Left circumflex. RCA Right coronary artery. IQR Interquartile range.

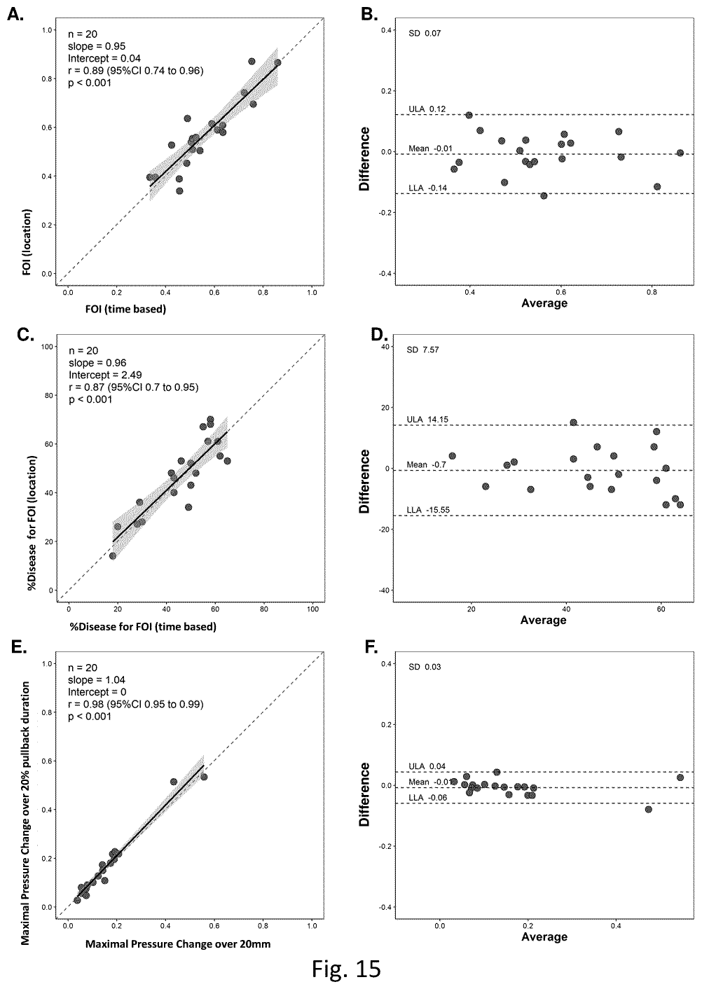

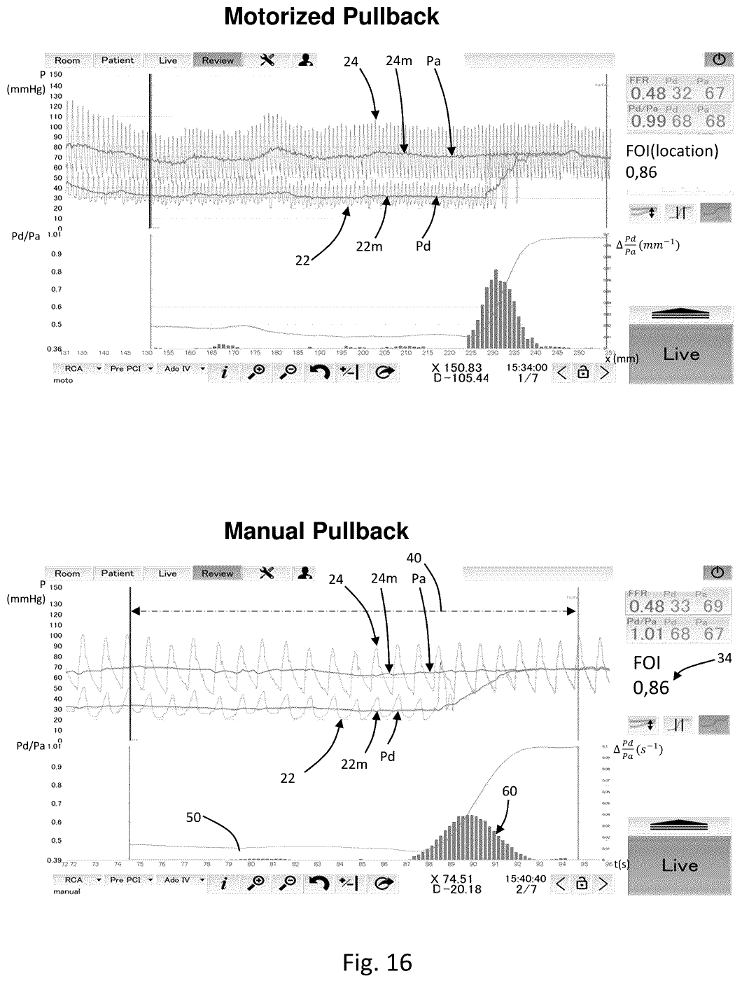

During a particular analysis based on the experiment, 20 vessels with both manual and motorized FFR pullbacks were analyzed. The mean FFR was 0.75±0.13 and 0.75±0.11 before manual and motorized pullbacks, respectively (mean difference 0.00±0.03, lower limit of agreement [LLA]−0.06 and upper limit of agreement [ULA] 0.07). The mean pullback duration were 32.1±76.1 for manual pullback and 103.3±24.8 seconds and motorized pullbacks, respectively. The mean time based FOI derived from manual pullback was 0.57±0.15 whereas the mean FOI(location) derived from motorized pullbacks was 0.56±0.14 (mean difference −0.01±0.07, LLA −0.14 and ULA 0.12) as shown in the table of FIG. 14, which is similar to the table of FIG. 7. As shown, the agreement on calculated FOI and its components between manual pullbacks for the new time based FOI and motorized pullbacks for FOI(location) is shown in FIG. 15. A case example is presented in FIG. 16, which shows a similar representation as the embodiment of FIGS. 4 and 5, in which a case example is shown of a patient from cohort 1. Motorized and manual pullbacks were performed in a right coronary artery. The calculated FOI was twice 0.86. It is however clear that for the motorized pullback the x-axis shows a position x in mm along the part of the vessel in which the pullback operation is performed.

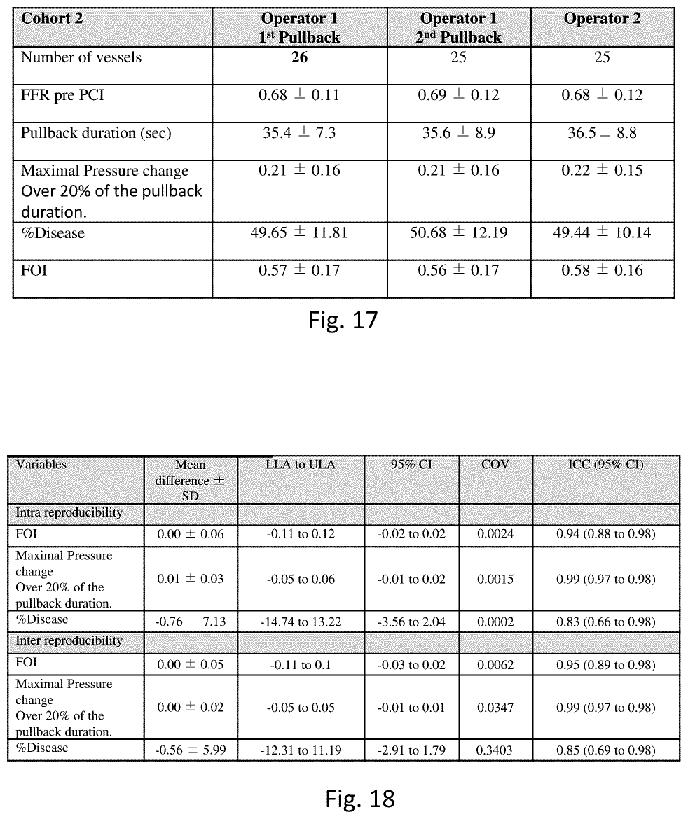

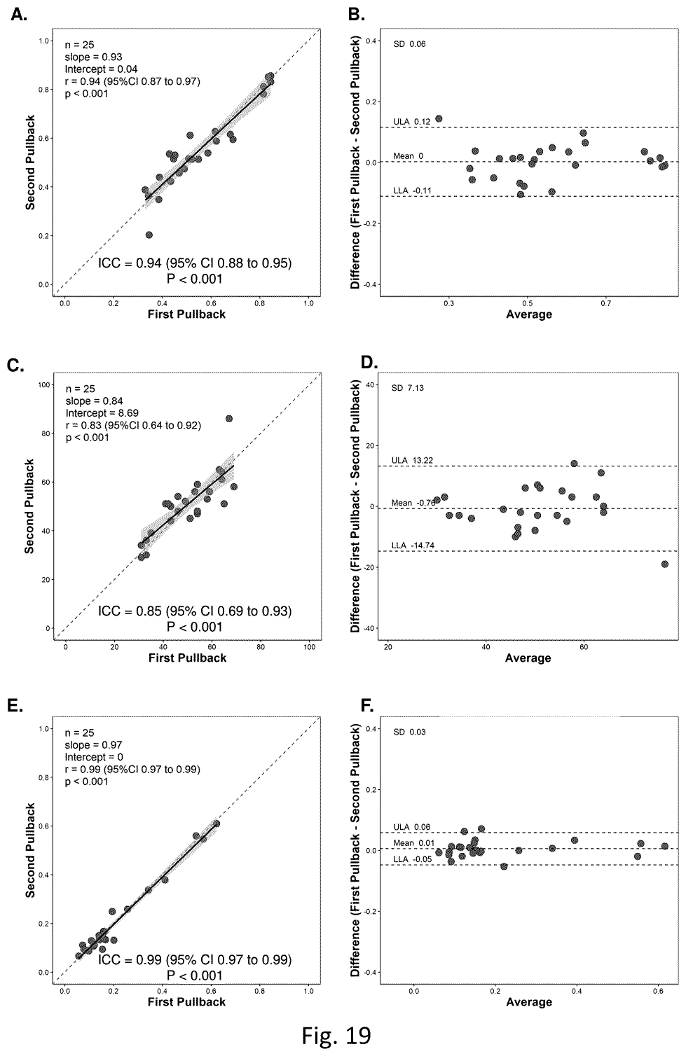

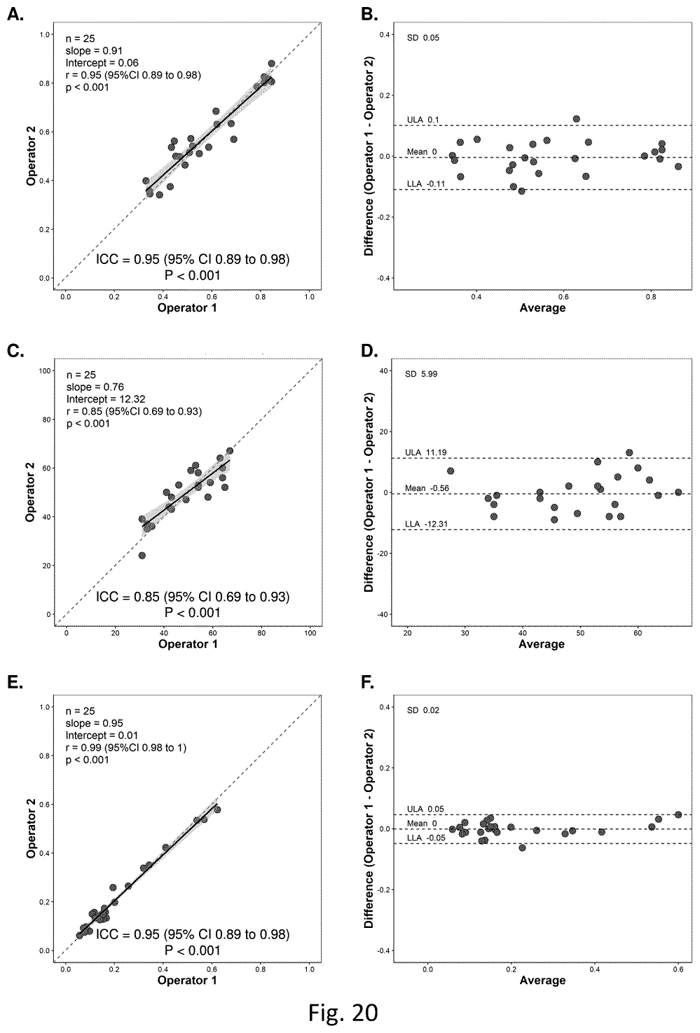

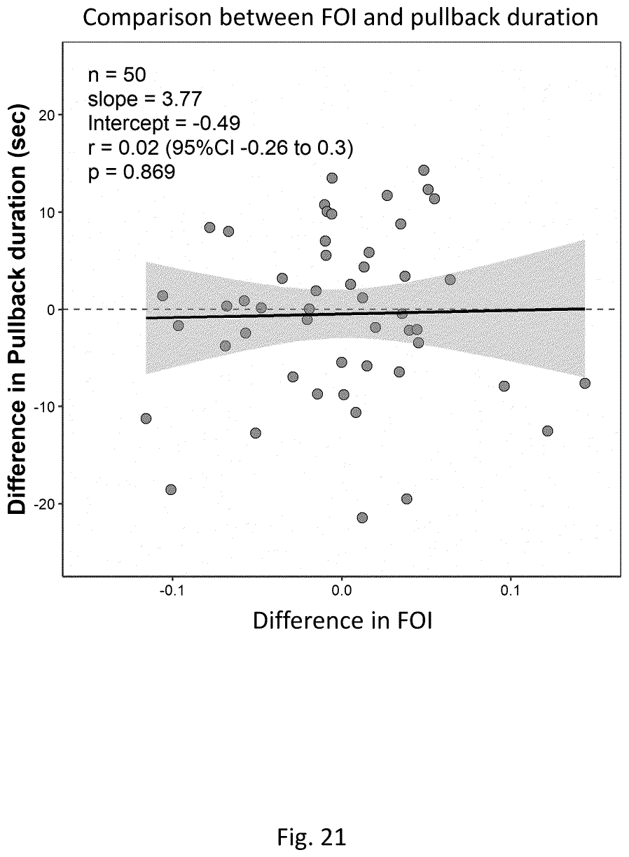

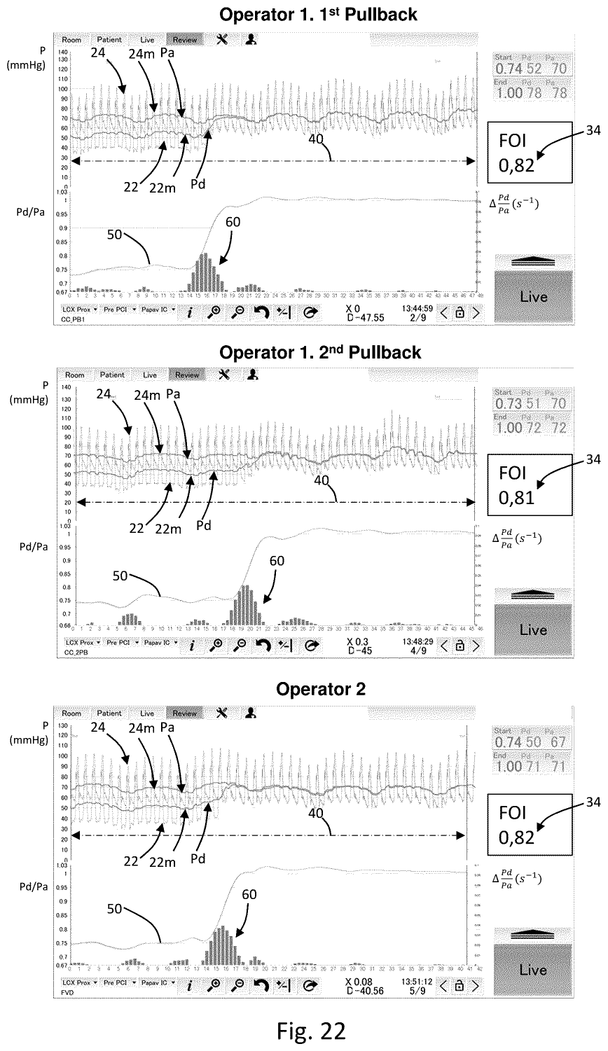

In a further analysis for assessing reproducibility of the time based FOI derived from manual pullbacks during the experiment, 26 vessels were included. In 25 vessels repeated manual FFR pullbacks were performed by one operator and in 25 vessels a third pullback was performed by a second operator. The mean FFR of the first, second and third measurement was 0.68±0.11, 0.69±0.12 and 0.68±0.12, respectively (p=0.920). The mean duration from the first, second and third pullback maneuver were 35.4±7.3 sec, 35.6±8.9 sec and 36.5±8.8 sec, respectively (p=0.924). The mean FOI derived from the first, second an third pullbacks were 0.57±0.17, 0.56±0.17 and 0.58±0.16, respectively as shown in the table of FIG. 17, which is similar to FIG. 7. There was surprisingly found an excellent intra-observer reproducibility of the FOI with a mean difference 0.00±0.06 (LOA −0.11, ULA 0.12), COV of 10.06% (95% CI 0 to 14.63) and intra class correlation (ICC) 0.94 (0.88 to 0.98) as shown in the table of FIG. 18. The reproducibility of the FOI and its components derived from the intra-observer analysis are shown in FIG. 19, which shows intra-operator agreement on the FOI (Panel A and B) and its components (percentage of disease panel C and D, Maximal Pressure Change over 20% pullback duration panel E and F) between repeated manual pullbacks. The inter-operator reproducibility was also surprisingly excellent with a mean difference between FOI of 0.00±0.05 (LOA −0.11, ULA 0.10), COV of 7.31% (95 CI 4.72 to 9.19) and ICC of 0.95 (95% CI from 0.89 to 0.98), which are illustrated by means of the table in FIG. 18 and FIG. 20, which shows inter-operator agreement on the FOI (Panel A and B) and its components (percentage of disease panel C and D, Maximal Pressure Change over 20% pullback duration panel E and F) between repeated manual pullbacks. The duration of the pullback did not affect the reproducibility of the FOI (r=0.02 (95% CI −0.26 to 0.3, p=0.869), as shown in FIG. 21, which shows the influence of pullback time on FOI reproducibility. A case example is shown in FIG. 22, which shows a similar representation as in the embodiments of FIGS. 4 and 5, in which a case example of a of a FFR pullback and FOI calculation in a left circumflex artery in a patient from cohort 2 is illustrated The FOI was derived from repeated manual pullbacks by a first operator (FOI 0.82 and 0.81) followed by a manual pullback performed by a second operator (FOI 0.82).

All the pullback procedures during the experiment were performed without complications. From all patients, 73% ( 30/41) underwent PCI without in-hospital adverse events. Eight patients were treated medically, and 3 patients were referred to coronary artery bypass grafting.

The findings of the present study can be summarized as follows: (1) the new time-based FOI can be accurately derived from manual pullbacks; (2) Both intra- and inter-operator reproducibility of the FOI derived from manual pullbacks were excellent, and (3) the duration of the pullback maneuver did not affect the reproducibility of the FOI.

Fractional flow reserve measured at the distal coronary artery captures the effect of cumulative pressure losses related to stenoses and diffuse disease. An FFR pullback provides a second dimension to the distal measurement by depicting the distribution and magnitude of pressure losses longitudinally across the coronary vessel. Motorized pullbacks are advantageous because they are operator independent and allows for the co-registration of the pressure data along with position information and the anatomical images. In contrast, manual pullback depend on the operator technique, the pullback speed may vary within the pullback and between operators. This can impact the FFR curve morphology, the quantification of pressure changes or gradients and the interpretation of the pattern of CAD. Nonetheless, manual pullbacks are practical to execute, reduce procedural time and can be done using intra-coronary hyperemic agents such as papaverine. The present study demonstrated that the new FOI can be derived from manual pullbacks resulting in similar values compared to a known FOI(location) based on motorized pullbacks with a mean difference of −0.01±0.07 FOI units. Moreover, the agreement on the quantification of maximal pressure or FFR changes or gradients between a manual and a motorized pullbacks was excellent −0.01±0.03 FFR units. The main reason for this is that by using a fraction of 20% of the duration of the pullback curve as a time window for calculating the FOI as described above, the FOI calculation overcomes issues related to different pullback speeds. Lastly, the evaluation of the extent of the vessel affected by disease, or % Disease, which in the new FOI is based on a threshold relative to a time based change of FFR, provides for similar results as a calculation that requires location or length based information with a mean difference of −0.70±7.57%.

It is known that fractional flow reserve is a highly repeatable metric, and likewise, that FFR pullbacks performed with a mechanized device have been shown to have excellent reproducibility. However, up till now it was believed that the morphology of a pullback curve is sensitive to variations in a manual pullback technique. A skilled person would be concerned that fast pullbacks maneuvers would likely mask pressure gradients and that similarly, pullback maneuvers with varying speeds may misrepresent the location and magnitude of pressure gradients. In the present study, operators were only instructed to performed manual pullback maneuvers aiming a completing the maneuver in 20 to 40 seconds while keeping a constant or substantially constant speed throughout the maneuver. Following these simple recommendations, surprisingly the new time based FOI calculated based on repeated manual pullbacks either by the same or a different operator resulted in similar values. Surprisingly, the unavoidable different manual pullback times and speed did not affect the reproducibility of the FOI.

The current analysis shows a high test re-test reliability of the new time based FOI as described in this application and surprisingly, excellent intra- and inter-observer reproducibility supporting the clinical application of the FOI. The calculation of the FOI can be implemented easily into a commercially available console, such as for example CoroFlow as manufactured by Coroventis Research, Uppsala, Sweden, or any other suitable data processing system. The online availability of the FOI after a pullback operation facilitates the quantification of the pattern of CAD (i.e., focal or diffuse) in clinical practice. According to a preferred embodiment, the FOI could be indicated once the hemodynamic significance of CAD has been confirmed. In this way the FOI permits a refined clinical decision making by informing about the appropriateness of PCI based on the pattern of CAD even for example in cases of serial lesions. Furthermore, the FOI informs about the likelihood to achieve functional revascularization with PCI. The interpretation of the FOI value is preferably performed in conjunction with the visual inspection of the pullback curve. This is particularly advantageous for the assessment of lesion specific pressure gradients. In patients with a low FOI, alternative options to PCI could be considered. In contrast, vessels with a high FOI are optimal candidates for PCI. Conclusions from the experimental results were, that Surprisingly, the new FOI can be derived from manual FFR pullbacks resulting in similar values compared to the known FOI(location) based on motorized pullbacks. The inter- and intra-operator reproducibility of the new FOI derived from manual pullback was excellent.

It is clear that in the context of this description reference is made to a millimeter of mercury, which is a manometric unit of pressure currently defined as 133.322387415 pascals, and which is denoted mmHg or mm Hg, and which is a unit routinely used in medicine and many other scientific fields.

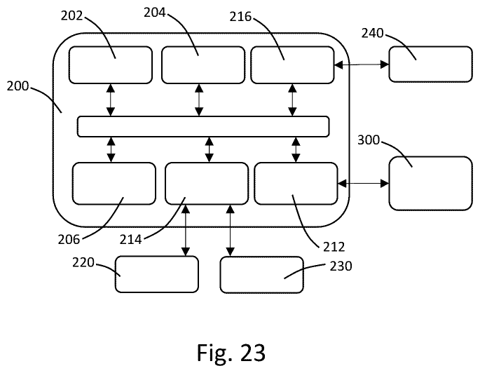

FIG. 23 schematically shows a suitable computing system 200, 300 for executing the computer-implemented method 100 described above. FIG. 23 thus shows a suitable computing system 200, 300 for hosting a data processing system or data acquisition system, comprising a processor configured to perform the computer-implemented method 100 or any of its components as described with reference to the above-mentioned embodiments. Computing system 200 for example comprises and/or consists of as a suitable general-purpose or application specific computer or integrated processing circuit and comprise a bus 210, a processor 202, a local memory 204, one or more optional input interfaces 214, one or more optional output interfaces 216, a communication interface 212, a storage element interface and one or more storage elements 206. Bus 210 may comprise one or more conductors that permit communication among the components of the computing system. Processor 202 may include any type of conventional processor or microprocessor that interprets and executes programming instructions. Local memory 204 may include a random-access memory (RAM) or another type of dynamic storage device that stores information and instructions for execution by processor 202 and/or a read only memory (ROM) or another type of static storage device that stores static information and instructions for use by processor 202. Input interface 214 may comprise one or more conventional mechanisms that permit an operator, sensors or other systems or devices to input information to the computing device 200, such as a keyboard 220, a mouse, a pen, voice recognition and/or biometric mechanisms, a data acquisition interface 230, a data acquisition system, etc. Output interface 216 may comprise one or more conventional mechanisms that output information to the operator, such as a display 240, a printer, a speaker, etc. Communication interface 212 may comprise one or more transceiver-like mechanisms such as for example one or more wired or wireless interfaces, such as for example Ethernet interfaces that enables computing system 200 to communicate with other devices and/or systems, for example mechanisms for communicating with one or more other computing systems 300. The communication interface 212 of computing system 200 may be connected to such another computing system 300 by means of a local area network (LAN) or a wide area network (WAN), such as for example the internet. Storage element interface may comprise a storage interface such as for example a Serial Advanced Technology Attachment (SATA) interface or a Small Computer System Interface (SCSI), or any other suitable storage interface for connecting bus 210 to one or more storage elements 206, such as one or more local disks, for example hard disk drives, flash storage devices, etc., and control the reading and writing of data to and/or from these storage elements 206. Although the storage elements 206 above is described as a local disk, in general any other suitable computer-readable media such as a removable magnetic disk, optical storage media such as a CD or DVD, -ROM disk, solid state drives, flash memory cards, . . . could be used.

The system according to the above-mentioned embodiments could be part of a suitable system running on a computing system 200 locally available to a user, such as a personal computer, laptop, a smart phone, a tablet computer, an electronic book reader, a portable computer, a desktop computer, etc. or on a remotely accessible computing system such as one or more servers available to a plurality of users. Alternatively, the system may also be part of suitable servers, for example comprising web-based tools. It is clear that, the system and the associated computer-implemented method, can be implemented as programming instructions stored in the local memory 204 of the computing system 200 for execution by its processor 202. Alternatively, these components could be stored on the storage element 206 or be accessible from another computing system 300 through the communication interface 212. In general, in this way the system and the associated computer-implemented method are provided as a computer program comprising software code adapted to perform this computer-implemented method when executed by a computing system. Alternatively, the system and the associated computer-implemented method could also be provided as a computer readable storage medium comprising computer-executable instructions which, when executed by a computing system, perform the computer-implemented method.

The present invention has been described with respect to particular embodiments and with reference to certain drawings, but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Unless defined otherwise, all technical and scientific terms used herein have the same meaning as commonly understood by one of ordinary skill in the art, e.g. in molecular biology, interventional cardiology fluid physics, biochemistry, and/or computational biology.

1. A data processing system comprising a processor, configured to:
receive a dataset comprising a set of pressure values measured during a pullback time period, the pullback time period corresponding to the time period during which the set of pressure values were determined from measurements of a movable pressure sensor while moving along a part of a vessel;
determine a time window of which the duration corresponds to a fraction of the pullback time period;
calculate the maximum of the moving time window pressure change based on the dataset of the part of the vessel.

2. The data processing system of claim 1, wherein the set of pressure values of the dataset, comprises:
pressure values determined from measurements of the movable pressure sensor while moving along the part of the vessel during the pullback time period;
optionally, pressure values determined from measurements of a stationary pressure sensor which remains stationary in the vessel during the pullback time period;
optionally, pressure values determined from a ratio of the measurements of the movable pressure sensor and the stationary pressure sensor during the pullback time period; and
optionally, a time-reference comprising:
an indication of the time during the pullback time period of the pressure measurements related to the set of pressure values;
an indication of a measurement frequency and/or a measurement interval of the pressure measurements related to the set of pressure values.

3. The data processing system claim 1, further configured to:
determine the duration of the time window in the range of 5% up to and including 50% of the pullback time period, wherein the duration of the pullback time period is in the range of:
at least 10 s; and/or
at least 10 heartbeat cycles; and/or
determine the duration of the time window in the range of:
at least 1 s; and/or
at least 1 heartbeat cycle.

4. The data processing system of claim 1, further configured to:
calculate from the dataset the pressure change associated with the pullback time period; and
calculate a ratio of:
the maximum pressure change associated with the time window; and
the pressure change associated with the pullback time period,
thereby determining the contribution of the pressure change during the time window with respect to the pressure change during the pullback time period.

5. The data processing system of claim 1, further configured to:
determine a threshold for a rate of change of the pressure values of the dataset in function of time;
determine, based on the dataset a portion of the pullback time period in which the rate of change is equal to or larger than the threshold.

6. The data processing system of claim 1, further configured to:
determine the threshold as a rate of change of the pressure values in the range of at least 0.05% of a maximum pressure value per subsection of the pullback time period.

7. The data processing system of claim 4, further configured to:
determine a threshold for a rate of change of the pressure values of the dataset in function of time;
determine, based on the dataset a portion of the pullback time period in which the rate of change is equal to or larger than the threshold; and
calculate a functional outcome index (FOI) based on the combination of:
the contribution of the maximum pressure change associated with the time window, with respect to the pressure change associated with the pullback time period; and
the portion of the pullback time period associated with the dataset in which the rate of change is equal to or larger than the threshold.

8. The processing system of claim 7, further configured to:
calculate the functional outcome index (FOI) based on the formula:
$$FOI = \frac{\begin{matrix}\frac{maximum pressure change ( time window )}{pressure change ( pullback time period )} + \\ ( 1 - threshold exceeding portion )\end{matrix}}{2} ,$$
wherein:
maximum pressure change (time window) is defined the maximum pressure change associated with the time window;
pressure change (pullback time period) is defined as the pressure change associated with the pullback time period; and
threshold exceeding portion is defined as the portion of the pullback time period associated with said dataset in which the rate of change is equal to or larger than the threshold.

9. The data processing system according to of claim 8, wherein
the set of relative pressure values comprises a fractional flow reserve (FFR) pullback curve comprising FFR values determined from measurements of the movable pressure sensor with respect to a stationary pressure sensor during the pullback time period,
and wherein the data processing system is further configured to:
calculate the functional outcome index (FOI) based on the formula:
$$FOI = \frac{\begin{matrix}\frac{maximum Δ FFR ( time window )}{Δ FFR ( pullback time period )} + \\ ( 1 - ( threshold exceeding portion ) )\end{matrix}}{2}$$
wherein:
maximum Δ FFR (time window) is defined as the maximum difference between FFR values associated with the start and the end of the time window;
Δ FFR (pullback time period) is defined as the difference between FFR values associated with the start and the end of the pullback time period; and
threshold exceeding portion is defined as the portion of the pullback time period associated with the dataset in which the rate of change in FFR is equal to or larger than the threshold.

10. The data processing system of claim 9, further configured to:
calculate the functional outcome index (FOI) such that the FOI is an expression of at least one of the following functional patterns of coronary artery disease:
a focal coronary artery disease;
a diffuse coronary artery disease.

11. The data processing system of claim 10, wherein, the data processing system is configured to output the value of the FOI such these values quantify at least one of the following functional patterns of coronary artery disease:
the functional pattern of a focal coronary artery disease when the value is higher than 0.7;
the functional pattern of a diffuse coronary artery disease when the value is lower than 0.4; and/or
functional pattern of a mixed coronary artery disease when the value is between 0.4 and 0.7.

12. The data processing system of claim 1, wherein the pressure values of the dataset are determined from pressure measurements along a part of a coronary vessel from a patient under hyperaemic conditions.

13. The data processing system of claim 1, wherein the data process system forms a portion of a larger system, wherein the larger system further comprises a data acquisition system coupled to and/or comprised in the data processing system, wherein the data acquisition system configured to:
receive as input the pressure measurements from the movable pressure sensor (22) when moved along the part of the vessel during the pullback time period wherein the movable pressure sensor is moved by means of at least one of:
a manual pullback of a pressure wire (28);
a motorized pullback of a pressure wire; and
generate and/or provide to the data processing system the set of pressure values of the dataset (32) based on the pressure measurements during the pullback time period; and optionally, a time-reference comprising:
an indication of the time during the pullback time period of the pressure measurements related to the set of pressure values;
an indication of a measurement frequency and/or a measurement interval of the pressure measurements related to the set of pressure values.

14. A system according to claim 13, wherein the data acquisition system further comprises at least one of the following comprising at least the movable pressure sensor and optionally a stationary pressure sensor:
A catheter comprising a pressure wire;
A catheter comprising a pressure wire configured for manual pullback along a predetermined part of a vessel during the pullback time period; and/or
A catheter comprising a pressure wire coupled to a motorized device with a fixed pullback speed during the pullback time period.

15. A computer-implemented method for operating the data processing system according to any of the preceding claims, wherein the method comprises the following steps performed by the data processing system:
receiving a dataset comprising a set of pressure values measured during a pullback time period, the pullback time period corresponding to the time period during which the set of pressure values were determined from measurements of a movable pressure sensor while moving along a part of a vessel;
determining a time window of which the duration corresponds to a fraction of the pullback time period;
calculating the maximum of the moving time window pressure change based on the dataset of the part of the vessel, and
wherein optionally the set of pressure values of the dataset relate to pressure measurements for a coronary vessel from a patient under hyperaemic conditions, and
wherein optionally the method comprises the further step of:
quantifying the patterns of a coronary artery functional disease in a coronary vessel from a patient under hyperaemic conditions.

16. The data processing system of claim 4, further configured to:
determine the threshold as a rate of change of the pressure values in the range of at least 0.05% of a maximum pressure value per subsection of the pullback time period;
calculate a functional outcome index (FOI) based on the combination of:
the contribution of the maximum pressure change (associated with the time window, with respect to the pressure change associated with the pullback time period; and
the portion of the pullback time period associated with the dataset in which the rate of change is equal to or larger than the threshold.

17. The processing system according to claim 16, further configured to:
calculate the functional outcome index (FOI) based on the formula:
$$FOI = \frac{\begin{matrix}\frac{maximum pressure change ( time window )}{pressure change ( pullback time period )} + \\ ( 1 - threshold exceeding portion )\end{matrix}}{2} ,$$
wherein:
maximum pressure change (time window) is defined the maximum pressure change associated with the time window;
pressure change (pullback time period) is defined as the pressure change associated with the pullback time period; and
threshold exceeding portion is defined as the portion of the pullback time period associated with the dataset in which the rate of change is equal to or larger than the threshold.

18. The data processing system according to claim 17, wherein
the set of relative pressure values comprises a fractional flow reserve (FFR) pullback curve comprising FFR values determined from measurements of the movable pressure sensor with respect to a stationary pressure sensor during the pullback time period,
and wherein the data processing system is further configured to:
calculate the functional outcome index (FOI) based on the formula:
$$FOI = \frac{\begin{matrix}\frac{maximum Δ FFR ( time window )}{Δ FFR ( pullback time period )} + \\ ( 1 - ( threshold exceeding portion ) )\end{matrix}}{2}$$
wherein:
maximum Δ FFR (time window) is defined as the maximum difference between FFR values associated with the start and the end of the time window;
Δ FFR (pullback time period) is defined as the difference between FFR values associated with the start and the end of the pullback time period; and
threshold exceeding portion is defined as the portion of the pullback time period associated with the dataset in which the rate of change in FFR is equal to or larger than the threshold.

19. The data processing system according to claim 18, further configured to:
calculate the functional outcome index (FOI) such that the FOI is an expression of at least one of the following functional patterns of coronary artery disease:
a focal coronary artery disease;
a diffuse coronary artery disease.

20. The data processing system according to claim 19, wherein, the data processing system is configured to output the value of the FOI such these values quantify at least one of the following functional patterns of coronary artery disease:
the functional pattern of a focal coronary artery disease when the value is higher than 0.7;
the functional pattern of a diffuse coronary artery disease when the value is lower than 0.4; and/or
functional pattern of a mixed coronary artery disease when the value is between 0.4 and 0.7.
